(19) United States
(12) Patent Application Publication
(10) Pub. No.: US 2023/0178196 A1
(43) Pub. Date: Jun. 8, 2023

(54) System, Method, and Software for Managing Digital Animal Records

(71) Applicant: Prox Animal Health, LLC, Fort Worth, TX (US)

(72) Inventor: Barry Beverly, Fort Worth, TX (US)

(21) Appl. No.: 18/061,440

(22) Filed: Dec. 3, 2022

Related U.S. Application Data
(60) Provisional application No. 63/264,918, filed on Dec. 3, 2021.

(51) Int. Cl. G16H 10/60; G06Q 10/087; A61B 6/00

(52) U.S. Cl. CPC G16H 10/60; G06Q 10/087; A61B 6/508; A61B 6/463

(57) ABSTRACT

In certain embodiments, a method includes accessing, in response to a first user input and via an equine records storage system, digital equine records for equines that include equine data for the equines. The equine data includes equine inventory data, certification data, and health data. The method includes generating, according to the equine inventory data and health data, an equine inventory user interface that includes identifying information of a first equine and health information for the first equine. The method includes accessing, in response to a second user input and via the storage system, the equine certification data, and generating, according to the equine certification data, an equine certifications user interface that includes certifications of the first equine. The method includes receiving, via the equine inventory user interface, equine inventory update data, and, in response, transmitting an equine inventory update to the storage system for updating the equine inventory data.

### CROSS-REFERENCE TO RELATED APPLICATIONS

This application claims the benefit of U.S. Provisional Application No. 63/264,918, filed on Dec. 3, 2021, which is incorporated by reference.

### TECHNICAL FIELD

This disclosure relates generally to software, and, in particular embodiments, to a system, method, and software for managing digital animal records.

### BACKGROUND

Farms, ranches, and the like often maintain hundreds if not thousands of animals for a variety of purposes. For example, family or corporate livestock operations may raise cows, pigs, chickens, horses, and/or other animals for one or more commercial purposes. In one example, the commercial purpose may include raising the animals for use in a type of event and/or for sale. As a particular example, an equine farm/ranch may raise horses for racing and/or for sale. As part of raising the animals (e.g., horses), workers associated with the farm may be responsible for keeping track of the animals and managing the health of the animals being raised on the farm. Managing the health of the animals may include managing the vaccinations given to the animals, managing any treatments given to the animals, managing the breeding of the female animals, and other suitable tasks.

### SUMMARY

In certain embodiments, a non-transitory computer-readable storage medium stores a program for execution by one or more processors. The program includes instructions for accessing, in response to a first user input and via an equine records storage system, digital equine records for equines that include equine data for the equines. The equine data includes equine inventory data, certification data, and health data. The program includes instructions for generating, according to the equine inventory data and health data, an equine inventory user interface that includes identifying information of a first equine and health information for the first equine. The program includes instructions for accessing, in response to a second user input and via the storage system, the equine certification data, and generating, according to the equine certification data, an equine certifications user interface that includes certifications of the first equine. The program includes instructions for receiving, via the equine inventory user interface, equine inventory update data, and, in response, transmitting an equine inventory update to the storage system for updating the equine inventory data.

In certain embodiments, a method includes accessing, in response to a first user input and via an equine records storage system, digital equine records for equines that include equine data for the equines. The equine data includes equine inventory data, certification data, and health data. The method includes generating, according to the equine inventory data and health data, an equine inventory user interface that includes identifying information of a first equine and health information for the first equine. The method includes accessing, in response to a second user input and via the storage system, the equine certification data, and generating, according to the equine certification data, an equine certifications user interface that includes certifications of the first equine. The method includes receiving, via the equine inventory user interface, equine inventory update data, and, in response, transmitting an equine inventory update to the storage system for updating the equine inventory data.

In certain embodiments, a system includes a storage system, one or more processors, and a non-transitory computer-readable storage medium storing a program to be executed by the one or more processors. The program includes instructions for receiving equine data for equines that includes equine inventory data, equine certification data, and equine health data. The program includes instructions for generating, in response to receiving the equine data for the equines, digital equine records on the storage system, the digital equine records including the equine data for the equines. The program includes instructions for receiving, from a first requesting device, a request for at least a portion of the equine inventory data for a first equine; accessing, from digital equine records on the storage system, the equine inventory data for the first equine; and transmitting, to the first requesting device, the at least a portion of the equine inventory data for the first equine for display by an application on the first requesting device. The program includes instructions for receiving, from a second requesting device, a request for at least a portion of the equine certification data for a second equine; accessing, from the digital equine records on the storage system, the equine certification data for the second equine; and transmitting, to the second requesting device, the at least a portion of the equine certification data for the second equine for display by an application on the second requesting device.

In certain embodiments, a non-transitory computer-readable storage medium stores a program for execution by one or more processors. The program includes instructions for accessing, in response to a first user input and via a storage system storing digital animal records for animals, the digital animal records for a first animal, the digital animal records including animal background data and animal health data. The program includes instructions for generating, according to the animal background data and the animal health data, an animal user interface that includes background information and animal health information for the first animal. The program includes instructions for receiving a prescription request for generating a new prescription for the first animal; and generating, in response to the prescription, a prescription user interface for generating the new prescription for the first animal, the new prescription being for a medication or a vaccination. The program includes instructions for receiving, via the prescription user interface, prescription information for the new prescription; and transmitting, to the storage system, a prescription update to update the digital animal records with the prescription information for the new prescription.

### BRIEF DESCRIPTION OF THE DRAWINGS

For a more complete understanding of this disclosure, and the advantages thereof, reference is now made to the following descriptions taken in conjunction with the accompanying drawings, in which:

FIG. 1 illustrates an example system for managing digital animal records for managing animal health, according to certain embodiments;

FIG. 2 illustrates an example user device, according to certain embodiments;

FIG. 3 illustrates an example processing system and associated storage unit, according to certain embodiments;

FIGS. 4A-4O illustrate example user interfaces that may be generated for managing animal health, according to certain embodiments;

FIGS. 5A-5R illustrate example user interfaces that may be generated for providing certain features for a veterinarian, according to certain embodiments;

FIG. 6 illustrates example digital animal records, according to certain embodiments;

FIG. 7 illustrates an example method for managing digital animal records for managing animal health, according to certain embodiments;

FIG. 8 illustrates an example method for managing digital animal records for managing animal health, according to certain embodiments; and

FIG. 9 illustrates an example method for managing digital animal records for managing animal health, according to certain embodiments.

### DETAILED DESCRIPTION OF ILLUSTRATIVE EMBODIMENTS

Managing the inventory and health of animals, such as horses, being raised for commercial or other purposes is a daunting task. Conventionally, the vast majority of inventory records and animal health procedures and records have been recorded by hand by farmers, horse owners, or other workers in the field, using a notepad for example, which may be not only unreliable, inefficient, and insecure, but may lack an ability for the health record to travel with an animal, either to a show/event (e.g., a race) or when the animal is sold, or otherwise be accessible in a variety of locations or to various entities (e.g., the horse owner and a veterinarian).

Equine farms still use paper records, which may present various problems. For example, a horse owner (or an associated employee, such as a ranch hand) may experience difficulty remembering to administer on-time treatments and/or vaccinations. As another example, communication between a veterinarian and the farm may be difficult, particularly after normal business hours. As another example, with paper records, no electronic medical record exists that can be shared in a digital format. As a particular example, when a horse is purchased at a sale, the medical record for the horse typically is unavailable for inspection by the buyer/buyer's veterinarian. As another example, when attending a show or race, the horse owner might forget appropriate association papers, the paper health certificate, or the paper membership card membership card (e.g., as may be required by the entity hosting the show or race). In an equine context, the inability to have accurate health records and other certifications that travel with the horse may present anti-doping issues in horse-race and horse-show animals. As another example, when a horse owner is traveling, it may be difficult or impossible to know that someone (whether an employee, friend, or other person) correctly fed and cared for the owner's horses back at the farm. As another example, the paper records used by equine farms may be inefficient on staffing and productivity, and may provide no practical way to see disease outbreak trends. Producers may lose track of animal health events and illness, potentially increasing costs. As another example, relying on printed reports within an entity (e.g., the family or company operating the equine farm) may lack an ability to perform/review immediate data analytics.

Even to the extent some or all of this information could be entered into an electronic spreadsheet, interacting with and managing a spreadsheet, let alone on a scale that might be associated with certain types of use cases, would be inconvenient, difficult, error-prone, and frustrating. Spreadsheets quickly become unmanageable and lack any intuitive interface for visualizing and interacting with the data. Furthermore, interacting with a spreadsheet can present other problems, such as access control, version management, and the like.

Thus, in general, paper records and simple spreadsheet-style electronic records suffer numerous deficiencies that can be overcome with digital animal records and one or more applications that provide user interfaces and functionality for interacting those digital animal records.

Embodiments of this disclosure provide a livestock animal health management software platform, which may promote animal health and reliable data recordation in horses or other types of animals/livestock. The software platform may be implemented, at least in part, as a software application (e.g., a mobile application or other suitable type of software application). At least a portion of the system may be implemented as a mobile application that can be downloaded by a user to a user device (e.g., a mobile device) of the user, such as a mobile smartphone or tablet computer. Users may include any suitable types of users, such as farmers/ranchers and associated users (e.g., employees or otherwise authorized users), veterinarians, or other suitable users.

An example implementation of an equine health monitoring application may be a mobile software platform for horse owners that includes horse profiles, vaccination/dewormer reminders/records, general health/medical records, a veterinarian video chat feature, an away care management feature, breeding records, registration certificates, and any other suitable features.

Certain embodiments provide a veterinarian application suitable for use by a veterinarian, potentially in conjunction with veterinary clients who use the above-described animal applications. The veterinarian application may be a mobile software platform for veterinarians. The platform may include vaccination/dewormer reminders/records, general health/medical digital record, vet video chat, breeding records, registration certificates, horse (or another animal type, if appropriate) profiles, and financial transaction functionality.

Embodiments of this disclosure provide a server system for generating and maintaining a database of inventory records and health records for animals (e.g., horses).

Embodiments of this disclosure provide an application for executing on user devices (e.g., of horse owners/farm operators and/or veterinarians) and that is configured to generate user interfaces for entering, accessing, and displaying animal data, including animal inventory data, animal health data, and the like. The user interfaces may facilitate entering and receiving data for updating the animal health records, and for sending the updates to data storage for maintaining a centralized digital health record for the animal. The user interface screens may provide a highly intuitive arrangement for accessing, managing, and viewing the animal health records.

The architecture described herein may be implemented in a computer or network of computers having one or more processors executing instructions of software programs that are stored in one or more computer-readable storage.

It should be understood that throughout this description and the accompanying claims, where applicable, the term “animal” may be replaced with a reference to a particular animal (e.g., “equine”).

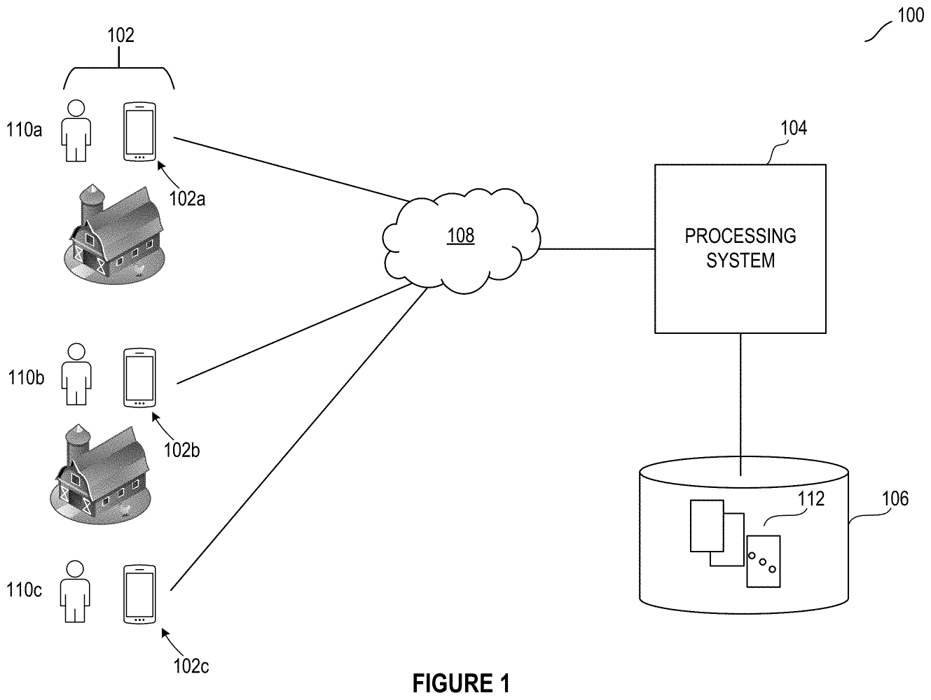

FIG. 1 illustrates an example system 100 for managing digital animal records for managing animal health, according to certain embodiments. In the illustrated example, system 100 includes user devices 102, processing system 104, storage unit 106, and network 108. In general, system 100 may be used to manage health of animals using digital animal records stored on storage unit 106 and via a software application installed on user devices 102.

User devices 102 may include any suitable computing device(s) capable of running software applications and accessing network 108. User devices 102 may be implemented in any suitable combination of hardware, firmware, and software. User devices 102 may include one or more computer systems at one or more locations. Each computer system may include any appropriate input devices, output devices, mass storage media, processors, memory, or other suitable components for receiving, processing, storing, and communicating data. For example, each computer system may include an integrated circuit (IC), printed circuit board (PCB), personal computer, laptop computer, workstation, network computer, kiosk, wireless data port, personal data assistant (PDA), one or more Internet Protocol (IP) telephones, one or more cellular/smart phones, tablet computers, wearable devices (e.g., smartwatch), one or more processors within these or other devices, or any other suitable processing device. Certain ones of the above or other user devices 102 may be mobile devices, such that they may be readily hand-carried by a user.

Users noa-noc (referred to generally as users no) of user devices 102 may include any suitable users. In certain embodiments, users no of user devices 102 may include farmers, employees of a farm or other livestock operation, veterinarians, or any other suitable users who may access or otherwise interact with digital animal records. In certain embodiments, at least a portion of users no of user devices 102 include personnel associated with raising animals. In the illustrated example, user noa of user device iota and user 110b of user device 102b represent users present on a farm who interact with the animals and access and otherwise interact with digital animal records via user devices iota and 102b. User noc of user device 102c represents a user who may be remote from the farm but still be able to access and otherwise interact with digital animal records 112 via user device 102c. Users noa-noc may be associated with the same farms or different farms, and those farms may raise the same or different types of animals. Furthermore, the farms might or might not be associated with one another from an ownership/management perspective.

Using user devices 102, users no may enter, access, modify, filter, and otherwise interact with digital animal records 112 stored by processing system 104. Software running on user devices 102 may provide a variety of functionality, including generating and displaying associated user interfaces, to allow users no of user devices 102 to enter, access, modify, filter, and otherwise interact with digital animal records 112 stored by processing system 104.

For example, user devices 102 may allow users no to enter animal data for animals to be managed using system 100, and to create digital animal records 112 to be stored by processing system 104. The animal data may include animal inventory data and/or animal health data. For example, users no of user devices 102 may enter various biographical information about the animals to be managed using system mo, such as an identifier, a sex, a birthdate, location information, or any other suitable information that may be useful in maintaining an inventory of animals to be managed using system 100. As another example, users no of user devices 102 may enter various health data about the animals to be managed using system 100, such as medical records, vaccination dates, health events, treatments, breeding records, or any other suitable health data that may be useful in maintaining the health of an inventory of animals to be managed using system 100.

As another example, using user devices 102 users no may search and filter digital animal records 112. For example, using user devices 102, users no may be able to access various user interface screens that provide views into different portions of the digital animal records 112. As a particular example, using user devices 102, users no may be able to view the entire population of animals being managed using system 100, filter down to a particular subset of animals being managed using system 100 (e.g., view certain information about the animals in a particular location), or view particular records for particular animals.

As another example, using user devices 102, users no may be able to set reminders for particular health events for the animals. For example, users no may be able to set reminders to administer particular vaccines or medications, schedule veterinary visits, inseminate female animals, and the like.

As another example, using user devices 102 users no may be able to manage the breeding of the female population of the animals being managed using system mo. For example, user devices 102 may allow users to maintain breeding schedules, view historical breeding records, manage embryo transfer records, view breeding records, set reminders associated with breeding, or perform other suitable tasks associated with managing the breeding of the female portion of the managed animal population.

As another example, using user devices 102 users no may be able to upload, view, and manage documentation associated the animals being managed using system 100. Such documentation may include birth certificates, vaccination records, veterinary visit summaries, or any other suitable documentation. In certain embodiments, users no may be able to upload videos, such as videos of veterinary visits.

As another example, using user devices 102 users no may be able to manage health events associated with the managed animal population. For example, users no may be able to record and track illnesses, track administration of treatments for those illnesses, recognize and follow trends to detect an outbreak of certain illnesses, and the like.

Example features that may be provided using user devices 102 may include digital electronic animal health records, digital electronic medical records, breeding records, embryo transfer records, veterinarian video chat, vaccination and medical reminders, an away care portal, a weather prediction application, digital animal inventory, dashboard analytics, and security. Certain embodiments allow a user to view, share, and/or update vaccination, medication (e.g., antibiotic), and dewormer records and reminders. Certain embodiments allow a user to view, share, and/or update photo-enhanced herd health, breeding, and heat cycle records. Certain embodiments allow a user (e.g., a farmer) to connect with a veterinarian, including potentially after normal business hours, via a video chat within the application. Certain embodiments allow users to share X-rays (e.g., a farmer to share an X-ray with a veterinarian or vice versa). Certain embodiments allow users to coordinate pasture and grass management. Certain embodiments facilitate maintaining farrier and dental health records.

Certain embodiments provide a dashboard feature. The dashboard feature may provide a broad view of the entire horse farm. The dashboard may show all horses and sort upcoming tasks/statuses such as pregnant, in-heat, and horses to be seen by farrier and/or veterinarian. Certain embodiments provide a farm veterinary record. The farm veterinary record may provide a summary screen of all veterinary activities for the farm. This summary screen may be a supplement to an individual electronic medical record for each animal. Certain embodiments may provide an individual horse inventory. A horse inventory screen for an individual horse may provide a summary of vaccination, medication, and health visits for the associated horse. This data can be synchronized with the veterinarian's records or sent to a local veterinarian while on the road. Certain embodiments provide for away care of animals. For example, away care may be useful for people who are away at shows or just away from their farm. Detailed care instructions may be sent to an employee, friend, neighbor, or other entity caring for the horses. Once associated tasks are completed, a notification (e.g., an in-app message) may be sent to the horse owner confirming that the tasks were completed according to the owner's instructions. Certain embodiments may provide a veterinarian video chat feature, which is an innovative way for an owner (or other appropriate user) to communicate with a veterinarian, possibly in an emergency setting, to determine whether it is appropriate for the veterinarian to make an in-person trip to care for the horse, potentially in the middle of the night. Certain embodiments reduce or eliminate paper records, transferring to completely digital format and producing an individual electronic medical record for each animal.

Processing system 104 may include any suitable computing device(s) operable to communicate with one or more user devices 102 through network 108 to facilitate managing animal health. Processing system 104 may be implemented in any suitable combination of hardware, firmware, and software. Processing system 104 may include one or more computer systems (e.g., one or more servers) at one or more locations. Each computer system may include any appropriate input devices, output devices, mass storage media, processors, memory, or other suitable components for receiving, processing, storing, and communicating data. Processing system 104 may include, for example, a file server, a domain name server, a proxy server, a web server, a computer workstation, or any other device operable to respond to requests for data from user devices 102 and/or manage storage and processing of data in storage unit 106. Processing system 104 may execute with any of the well-known MS-DOS, PC-DOS, OS-2, MAC-OS, WINDOWS, UNIX, or other appropriate operating systems, including future operating systems.

Processing system 104 may include one or more software applications that communicate with a software application running on user devices 102 and that facilitate creating, maintaining, and searching of digital animal records 112 in storage unit 106.

Storage unit 106 may include any number of tangible storage media. Additionally, all or part of storage unit 106 could reside locally within processing system 104 or could reside at a location external but accessible to processing system 104 (e.g., within removable media, and/or at an external database). In certain embodiments, storage unit 106 may take the form of a suitable combination of volatile and non-volatile memory including, without limitation, magnetic media, optical media, random access memory (RAM), read only memory (ROM), removable media, or any other suitable memory component. Where appropriate, RAM may be dynamic RAM (DRAM) or static RAM (SRAM). Moreover, where appropriate, this RAM may be single-ported or multi-ported RAM. This disclosure contemplates any suitable RAM. In certain embodiments, storage unit 106 may include one or more databases. In certain embodiments, a portion of all of storage unit 106 may include a database, such as one or more structured query language (SQL) servers or relational databases. In certain embodiments, a portion or all of storage unit 106 may implement a portion or all of a blockchain. The one or more storage units 106 may be implemented using one or more computer systems/servers and may be connected to a network via which user devices may be able to access/update associated data records.

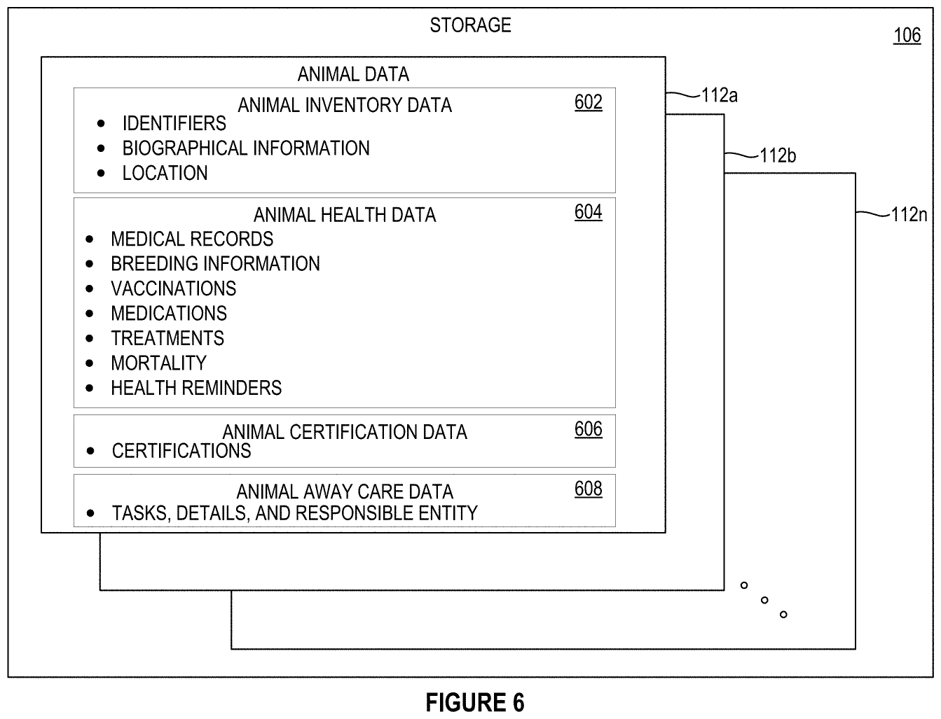

Storage unit 106 may be used to store digital animal records 112 that contain information that facilitates managing animal health of a plurality of animals. In certain embodiments, digital animal records 112 may include animal data, which may include animal inventory data, animal health data, any combination of the preceding, or other data that may facilitate managing animal health of a plurality of animals. Additional detail regarding digital animal records 112 is described below with reference to FIG. 6.

As just one example, embodiments may store collected data on one or more networked storage devices (e.g., in the so-called cloud). Certain embodiments use human-grade Health Insurance Portability and Accountability Act of 1996 (HIPAA)-compliant security for privacy. In certain embodiments, both users associated with a farm and one or more authorized veterinarians may be able to update/access at least some of the same data records as stored on the network so that such users are viewing or otherwise interacting with synchronized data.

User devices 102 may access features provided by processing system 104 and/or digital animal records 112 stored using storage unit 106 via network 108. Network 108 may include wireless and/or wireline platforms operable to exchange electronic data or information. In certain embodiments, network 108 may include one or more packet data networks offering communication interfaces or exchanges between components of system 100. Network 108 include any suitable combination of local area networks (LANs), metropolitan area networks (MANs), wide area networks (WANs), wireless local area networks (WLANs), virtual private networks (VPNs), intranets, Internets, plain old telephone systems (POTSs), or any other appropriate architecture or system that facilitates electronic communications. Certain networks 108 include wireless technology such as WiMax (802.16), WiFi (802.11), 3G, 4G, 5G, or any other suitable wireless technologies in any suitable combination.

Although particular animals may be described, this disclosure contemplate using any of the described features being used for any suitable type of animal/farm. For example, a cow or swine application may be implemented having one or more features similar to those described herein with reference to the equine application.

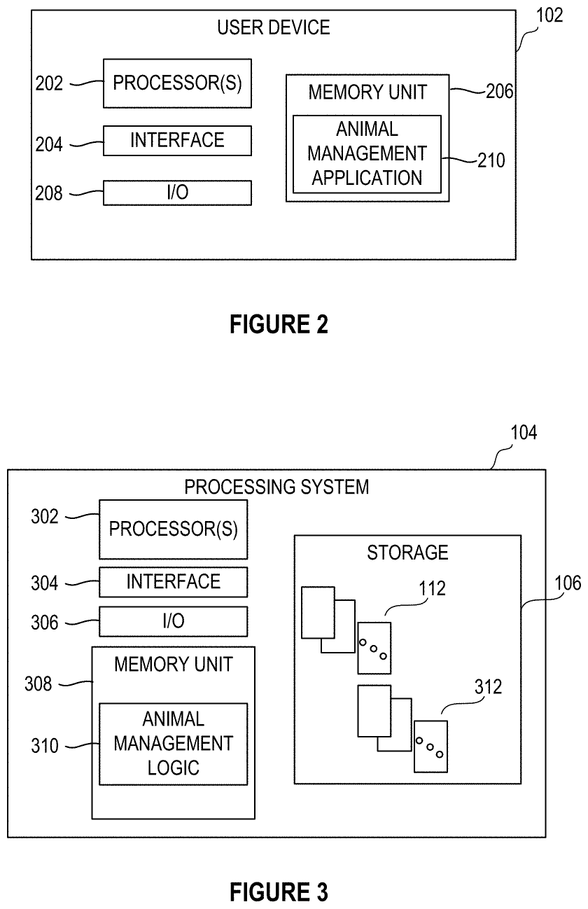

FIG. 2 illustrates an example user device 102, according to certain embodiments. In the illustrated example, user device 102 includes one or more processors 202, one or more interfaces 204, one or more memory units 206, and various I/O functionality 208, each referred to hereinafter in the singular for simplicity. In certain embodiments, user device 102 includes a combination of hardware, firmware, and software that facilitates managing animal health.

Processor 202 may refer, for example, to the one or more tangible devices of user device 102 capable of carrying out instructions, such as that of a computer program. For example, processor 202 may include one or more microprocessors, controllers, or any other suitable computing devices or resources. Processor 202 may work, either alone or with other components of user device 102, to provide a portion or all of the functionality of user device 102 described herein. For example, processor 202 may be execute or realize various logic-based functions of animal management application 210.

Interface 204 may include, for example, any tangible device(s) that may be used (e.g., by a person, or by another device or system) to communicate with user device 102. For example, any suitable combination of keypads, touch screens, and speech recognition devices may be considered input interfaces 204 and a display screen may be considered output interfaces 204.

Memory unit 206 may include any number of tangible storage media. Additionally, all or part of memory unit 206 could reside locally within user device 102 or could reside at a location external but accessible to user device 102 (e.g., within removable media, and/or at an external database). In certain embodiments, memory unit 206 may take the form of a suitable combination of volatile and non-volatile memory including, without limitation, magnetic media, optical media, RAM, ROM, removable media, or any other suitable memory component. Where appropriate, RAM may be DRAM or SRAM. This disclosure contemplates any suitable RAM. Memory unit 206 may include, for example, applications programs, network, communication programs, operating system software data, etc.

I/O functionality 208 may include, for example, any communication method of user device 102 (e.g., from an image buffer to a display). Input functionality may include, for example, the receiving of signals or data by user device 102. Output functionality may include, for example, the communication of signals or data by user device 102. These terms may also refer to part of an action, such as, for example, to “perform I/O” may involve performing an input and/or output operation. In some cases, one or more communication devices of user device 102 may be used to implement both input and output functionality.

User device 102 may include, or may be configured to access, animal management application 210 that when executed facilitates managing animal health. Animal management application 210 may be implemented in any suitable combination of hardware, firmware, and software. In certain embodiments, animal management application 210 includes a set of computer-readable instructions (e.g., stored in memory unit 206 or some other suitable computer-readable storage medium) that when executed by processor 202 are operable to perform certain operations.

Animal management application 210 may generate user interfaces and provide associated functionality for managing digital animal records 112.

Animal management application 210 may be web-based, for example, with parts of each page being loaded from a server and/or a database, or may be natively-compiled to execute on user device 102 even when no network is available. Although primarily described as a software application running on user device 102, this disclosure contemplates animal management application 210 being implemented as a set of web pages accessible in a web browser, for example.

In operation of an example embodiment of user device 102, animal management application 210 may access, in response to user input using user device 102 and via an animal records storage system (e.g., storage unit 106), digital animal records 112 for a plurality of animals. Digital animal records 112 may store animal data for the animals, and the animal data may include animal inventory data, animal certification data, and animal health data.

Animal management application 210 may generate, according to the animal inventory data and the animal health data, an animal inventory user interface. The animal inventory user interface may include identifying information of a first animal being managed using system 100 and health information of the first animal.

In certain embodiments, a user interface generated by animal management application 210 includes a link for adding a new animal to the inventory of managed animals. In such an example, animal management application 210 may be configured to receive animal inventory data for the new animal and transmit the animal inventory data for the new animal for storage in the animal records storage system (e.g., in digital animal records 112 of storage unit 106).

Animal management application 210 may access, in response to user input from user device 102 and via the animal records storage system (e.g., storage unit 106), animal certifications data. Animal management application 210 may generate, according to the animal certification data, an animal certifications user interface for managing the animal certifications of one or more of the animals. In certain embodiments, the animal certifications user interface may include certifications of the first animal.

Animal management application 210 may receive, via the animal inventory user interface displayed on user device 102, animal inventory update data. Animal management application 210 may transmit, in response to the animal inventory update data received via the animal inventory user interface, an animal inventory update to the animal records storage system (e.g., storage unit 106) for updating digital animal records 112 (e.g., animal inventory data).

Animal management application 210 may receive, via the animal certifications user interface displayed on user device 102, animal certifications update data. Animal management application 210 may transmit, in response to the animal certifications update data received via the animal certifications user interface, an animal certifications update to the animal records storage system (e.g., storage unit 106) for updating digital animal records 112 (e.g., animal certifications data).

In certain embodiments, animal management application 210 may generate a dashboard user interface screen that includes selectable links. Each selectable link may correspond to an associated feature and data set for managing animal health of the managed animal inventory. For example, a first selectable link may include a link to the dashboard user interface. As another example, a second selectable link may include a link to an away care user interface. As another example, a third selectable link may include a link to a veterinarian video chat user interface. As another example, a fourth selectable link may include a link to a health reminders user interface. As another example a fifth selectable link may include a link to a breeding records user interface. As another example, a sixth selectable link may include a link to a medical records user interface. As another example, a seventh selectable link may include a link to a racing user interface.

In certain embodiments, animal management application 210 is configured to access, via the animal records storage system (e.g., storage unit 106), breeding information for a subset of the animals, and to generating, according to the breeding information, a breeding user interface. As examples, the breeding user interface may include breeding information for the subset of animals or the breeding user interface may include breeding details for a particular animal of the subset of animals.

In certain embodiments, animal management application 210 is configured to access, in response to user input, animal away care data for one or more animals. The animal away care data may include tasks, task details, and a respective responsible entity for performing each task according to the task details for that task. Animal management application 210 may be configured to display, in response to accessing the animal away care data, at least a portion of the animal away care data for the one or more animals, and to generate, for each of the one or more animals, a respective notification that the tasks for the animal have been completed.

In certain embodiments, animal management application 210 is configured to receive user input requesting a video chat with a selected veterinarian; initiate, in response to the user input, the video chat with the selected veterinarian; and display a video chat user interface for conducting the video chat with the selected veterinarian.

In certain embodiments, animal management application 210 is configured to access, in response to user input, the animal health data for the first animal. The animal health data may include medical records for the first animal. Animal management application 210 may be configured to display, in response to the user input, at least a portion of the medical records for the first animal in a medical records user interface.

In certain embodiments, such as in certain equine contexts, the certifications available via the animal certifications user interface and/or the medical records available via the medical records user interface may provide a racing feature set and associated user interfaces for animal management application 210. For example, a user may be able to present or transmit, using animal management application 210, certain document to satisfy requirement for entering a horse (or other animal) in a race, in a show, or at another suitable event using the certifications and medical records available using animal management application.

In certain embodiments, animal management application 210 may implement one or more features for a veterinarian. For example, the veterinarian features and associated user interfaces provided by animal management application 210 may include a dashboard feature, an appointments feature, a medical records feature (e.g., for viewing medical records of animals of a client), a video chat feature, an image upload and sharing feature (e.g., for uploading and sharing X-ray images), a notifications feature, a settings feature, a calendar feature, a payments feature, and/or any other suitable features.

In operation of an example embodiment of user device 102, animal management application 210 may access, in response to user input using user device 102 and via an animal records storage system (e.g., storage unit 106) that stores digital animal records 112 for a plurality of animals, digital animal records 112 for a first animal. Digital animal records 112 may store animal data for the animals, and the animal data may include animal background data and animal health data.

Animal management application 210 may generate, according to the animal background data and the animal health data, an animal user interface. The animal user interface may include animal background information for the first animal being managed using system 100 and animal health information for the first animal. In certain embodiments, a user interface generated by animal management application 210 includes a link for adding a new animal to the inventory of managed animals. In such an example, animal management application 210 may be configured to receive animal inventory data for the new animal and transmit the animal inventory data for the new animal for storage in the animal records storage system (e.g., in digital animal records 112 of storage unit 106).

Animal management application 210 may receive, via the animal inventory user interface displayed on user device 102, a prescription request for generating a new prescription for the first animal. Animal management application 210 may generate, in response to the prescription request, a prescription user interface for generating the new prescription for the first animal. In certain embodiments, the new prescription may be for a medication or a vaccination.

Animal management application 210 may receive, via the prescription user interface (e.g., user interface 500e), prescription information for the new prescription. Animal management application 210 may transmit, to the storage system (e.g., storage unit 106), a prescription update to update digital animal records 112 with the prescription information for the new prescription.

Animal management application 210 may receive a video chat request for a video chat with a veterinarian, and animal management application 210 may display a video chat user interface for conducting the video chat between the veterinarian and a client (e.g., a horse owner).

Animal management application 210 may receive a second input selecting a selectable link to an appointments user interface. Animal management application 210 may access, in response to receiving the second input and via the storage system (e.g., storage unit 106), appointments data. Animal management application 210 may generate, in accordance with the appointments data, an appointments user interface. In certain embodiments, the appointments user interface may include filters for filtering a listing of appointments and the listing of appointments according to a selection of one or more of the filters.

In certain embodiments, animal management application 210 may generate a dashboard user interface screen that includes selectable links. In certain embodiments, these selectable links may be implemented, in part, using a dock. Each selectable link may correspond to an associated feature and data set for managing animal health of the managed animal inventory. For example, a first selectable link may include a link to the dashboard user interface. As another example, a second selectable link may include a link to an appointments user interface. As another example, a third selectable link may include a link to a medical records user interface. As another example, a fourth selectable link may include a link to a calendar user interface. As another example a fifth selectable link may include a link to a settings user interface.

In certain embodiments, the first input described above is a selection of the selectable link to the medical records user interface such that the animal user interface generated above is the medical records user interface, as generated for the first animal. The medical records user interface may include a first link, selection of which initiates generation of a vaccination user interface that is configured to list vaccinations for the first animal; a second link, selection of which initiates generation of a radiograph user interface that is configured to display an image of an X-ray for the first animal; and a third link, selection of which initiates generation of a surgery user interface that is configured to display notes or images associated with a surgery for the first animal.

In certain embodiments, a single animal management application 210 provides features both for a horse owner/operator and for a veterinarian. In certain other embodiments, different versions of animal management application 210 may exist. For example, a first version of animal management application 210 may provide features for use by a horse owner/operator, and a second version of the animal management application 210 may provide features for use by veterinarians. In such an example, one possibility is that horse owners/operators may download and use the first version of animal management application 210, while veterinarians may download use the second version of animal management application 210. It should be understood that in this instance, the term “version” might not refer to updates to animal management application 210, and instead might refer to the target users for the features provided by that version of animal management application 210.

Regardless of whether separate applications are used for animal management application 210, in certain embodiments, the features for the horse owner/operator and the features for the veterinarian may access at least some of the same information from digital animal records 112, which may reflect at least some of the benefits of the digital animal records 112 and associated user interfaces provided by animal management application 210 of certain embodiments of this disclosure.

FIG. 3 illustrates an example processing system 104 and associated storage unit 106, according to certain embodiments. In the illustrated example, processing system 104 includes one or more processors 302, one or more interfaces 304, various I/O functionality 306, one or more memory units 308, and storage unit 106, each referred to hereinafter in the singular for simplicity. In certain embodiments, processing system 104 includes a combination of hardware, firmware, and software that facilitates managing animal health.

Processor 302 may refer, for example, to the one or more tangible devices of processing system 104 capable of carrying out instructions, such as that of a computer program. For example, processor 302 may include one or more microprocessors, controllers, or any other suitable computing devices or resources. Processor 302 may work, either alone or with other components of processing system 104, to provide a portion or all of the functionality of processing system 104 described herein. For example, processor 302 may execute or realize various logic-based functions of animal management logic 310.

Interface 304 may include, for example, any tangible device(s) that may be used (e.g., by a person, or by another device or system) to communicate with processing system 104. In certain embodiments, any suitable combination of keyboards, mice, touch screens, and speech recognition devices may be considered an input interfaces 304 and monitors and printers may be considered output interfaces 304.

I/O functionality 306 may include, for example, any communication method of processing system 104 (e.g., from an image buffer to a display). Input functionality may include, for example, the receiving signals or data by processing system 104. Output functionality may include, for example, the communication of signals or data by processing system 104. These terms may also refer to part of an action, such as, for example, to “perform I/O” may involve performing an input and/or output operation. In some cases, one or more communication devices of processing system 104 may be used to implement both input and output functionality.

Memory unit 308 may include any number of tangible storage media. Additionally, all or part of memory unit 308 could reside locally within processing system 104 or could reside at a location external but accessible to processing system 104 (e.g., within removable media, and/or at an external database). In certain embodiments, memory unit 308 may take the form of a suitable combination of volatile and non-volatile memory including, without limitation, magnetic media, optical media, RAM, ROM, removable media, or any other suitable memory component. Where appropriate, RAM may be DRAM or SRAM. This disclosure contemplates any suitable RAM. Memory unit 308 may include, for example, applications programs, network, communication programs, operating system software data, etc.

Processing system 104 may include, or may be configured to access, animal management logic 310 that when executed facilitates managing animal health. Animal management logic 310 may be implemented in any suitable combination of hardware, firmware, and software. In certain embodiments, animal management logic 310 includes a set of computer-readable instructions (e.g., stored in memory unit 308 or some other suitable computer-readable storage medium) that when executed by processor 302 are operable to perform certain operations. In this example, animal management logic 310 is stored within memory unit 308; however, animal management logic 310 may be stored external to memory unit 308 and/or external to processing system 104.

Animal management logic 310 is an application that communicates with animal management applications 210 on user devices 102 and that facilitates creating, maintaining, and searching of digital animal records 112 in storage unit 106.

In certain embodiments, animal management logic 310 may enforce one or more security measures for restricting access, potentially with different tiers of access and operation capabilities, to digital animal records 112 stored on storage unit 106. Thus, users no may register with processing system 104, and animal management logic 310 of processing system 104 may manage the rights of those users to view (or not view), edit (or not edit), or otherwise interact with digital animal records 112.

The description of storage unit 106 from FIG. 1 is incorporated by reference. Additionally, all or part of storage unit 106 could reside locally within processing system 104 or could reside at a location external but accessible to processing system 104 (e.g., within removable media, and/or at an external database).

As described above, storage unit 106 may be used to store digital animal records 112 that contain data for managing animal health. In certain embodiments, digital animal records 112 may include animal data, which may include animal inventory data, animal health data, any combination of the preceding, or other information that may facilitate managing animal health. For example, the animal inventory data may include various biographical information about the source animals, such as an identifier, a sex, a birthdate, location information, or any other suitable information that may be useful in maintaining an inventory of source animals. As another example, health data about the source animals may include medical records, vaccination dates, health events, treatments, breeding records, or any other suitable health information. Additional detail regarding example digital animal records 112 are described below with reference to FIG. 6.

In certain embodiments, storage unit 106 may store digital veterinarian records 312 that contain data for use by veterinarian features of animal management application 210. In certain embodiments, digital veterinarian records may include account data, animal patient data, appointment data, payment data, and/or any other suitable data.

Account data may include information identifying a veterinarian (or set of veterinarians if, for example, a group of veterinarians work together under a single account) associated with the account, any passwords, certifications for the veterinarian, contact information, or other information associated with the account.

Animal patient data may include information regarding the biographical information for animals that are patients of the veterinarian, contact information for the owner of the animal patients, location information for the animal patients, animal health data for the animal patients, and/or any other suitable information regarding the animal patients.

In the case of the biographical information for the animal patient, such biographical information may include a name, birthdate, sex, animal type (broadly and/or specific breed), registration information for any entities with which the animal is registered, and/or any other suitable information.

In the case of the animal health data for the animal patient, such animal health data may include health reminders (vaccinations, medications, and the like), medical records (vaccinations, medications, and the like), certifications and other documentation, radiology information (e.g., X-Rays), surgery information, breed records information, and/or any other suitable information. In certain embodiments, a portion of digital animal records 112 and digital veterinarian records 312 may overlap, so that the horse owner features of animal management application 210 and the veterinarian features of animal management application 210 interact with the same data. In certain embodiments, this may provide certain advantages, particularly in connection with the animal health data.

Appointment data may include information regarding the appointments the veterinarian has or has previously had with animal patients, including scheduled appointments, pending appointments (not accepted or rejected), rejected appointments; appointment details (data, time, and the like); animal patient information associated with the appointments; appointment availability information indicating dates/times (potentially in blocks) that the veterinarian is available for appointments; and/or any other suitable information regarding appointments.

Payment data may include payment details for the veterinarian, including payments received, payments due, payment status, dates associated with payments, bank account information for transferring payments to a bank or to a different bank, and/or any other suitable information useful for managing the financial aspects associated with operating the veterinarian business.

In certain embodiments, the described processing and memory elements (such as processor(s) 302, memory unit 308, and storage unit 106) may be distributed across multiple devices such that the operations performed utilizing these elements may also be distributed across multiple devices. For example, software operated utilizing these elements may be run across multiple computers that contain these processing and memory elements. Other variations aside from the stated example are contemplated involving the use of distributed computing.

In operation of an example embodiment of processing system 104, animal management logic 310 may receive animal data for animals. The animal data may include animal inventory data, animal certification data, and animal health data. Animal management logic 310 may generate, in response to receiving the animal data for the animals, digital animal records 112 on the storage system (e.g., storage unit 106). Digital animal records 112 may include the animal data for the animals.

Animal management logic 310 may receive, from a requesting user device 102, a request for the animal inventory data for a first animal. Animal management logic 310 may access, from digital animal records 112 on the storage system (e.g., storage unit 106), the animal inventory data for the first animal. Animal management logic 310 may transmit, to the requesting user device 102, the animal inventory data for display by an application (e.g., animal management application 210) running on the requesting user device 102. In certain embodiments, the animal inventory data includes the following information for the managed inventory of animals: an animal identifier; animal type; and location information.

Animal management logic 310 may receive, from a requesting user device 102, a request for at least a portion of the animal health data for a subset of the animals. Animal management logic 310 may access, from digital animal records 112 on the storage system (e.g., storage unit 106), the animal health data for the subset of the animals. Animal management logic 310 may transmit, to the requesting user device 102, the animal health data for the subset of the animals for display by an application (e.g., animal management application 210) running on the requesting user device 102. In certain embodiments, the animal health data includes the following information for the managed inventory of animals: digital medical records; vaccination information; breeding information; and treatment information. In certain embodiments, the animal health data includes a schedule of health events and animal management logic 310 is configured to communicate a reminder of a health event to an application (e.g., animal management application 210) running on a requesting user device 102.

[moo] Animal management logic 310 may receive, from a requesting user device 102, a request for at least a portion of the animal certification data for a second animal (which may be the same or different than the first animal). Animal management logic 310 may access, from digital animal records 112 on the storage system (e.g., storage unit 106), the at least a portion of the animal certification data for the second animal. Animal management logic 310 may transmit, to the requesting user device 102, the at least a portion of the animal certification data for the second animal for display by an application (e.g., animal management application 210) running on the requesting user device 102.

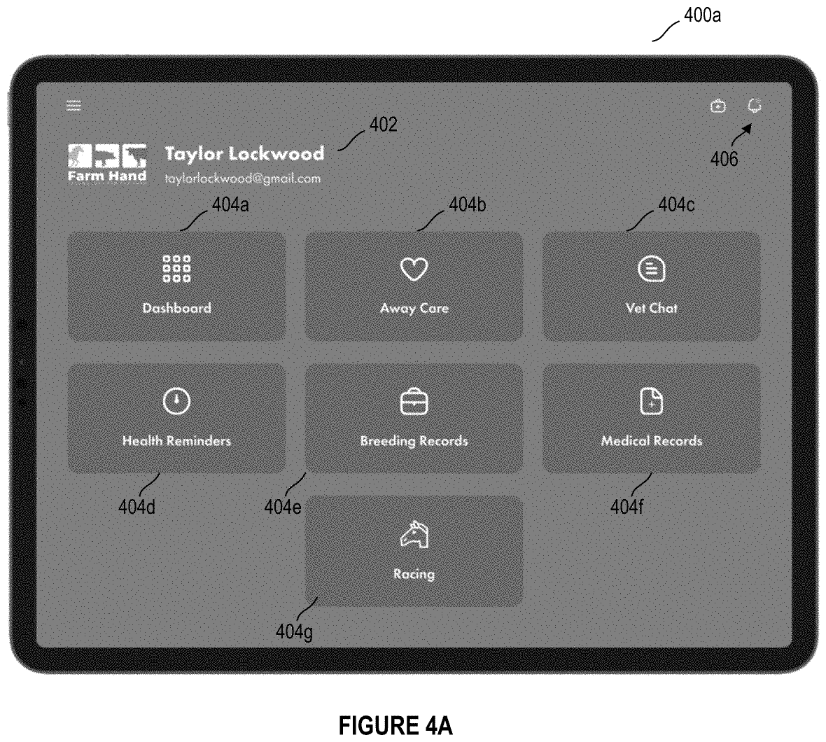

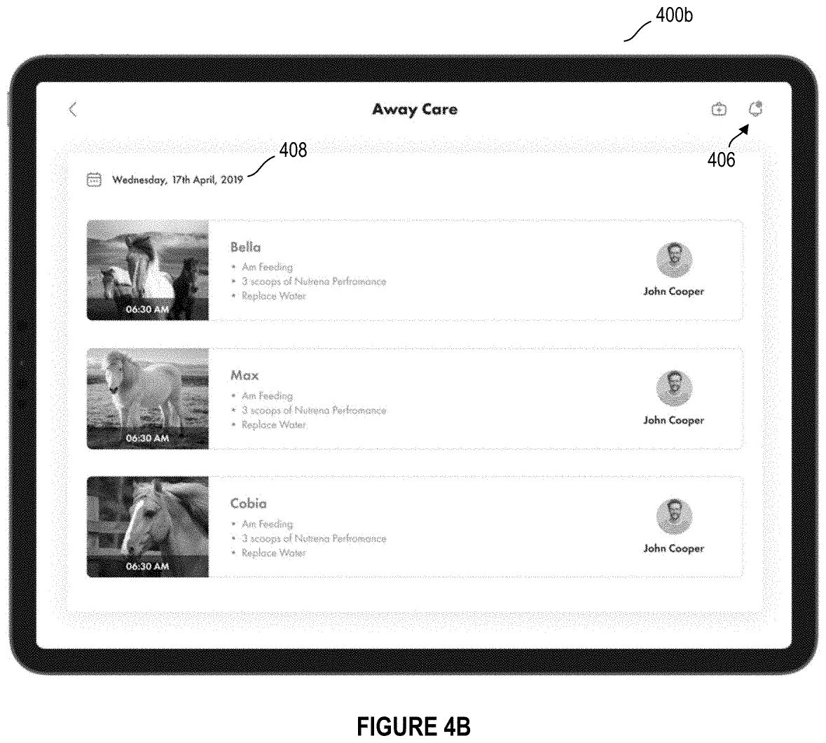

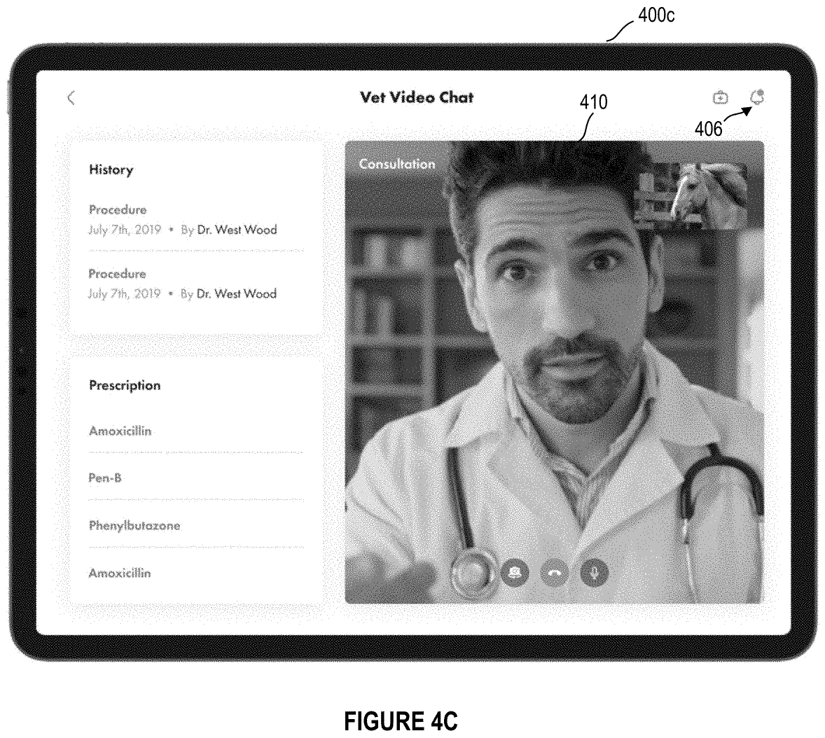

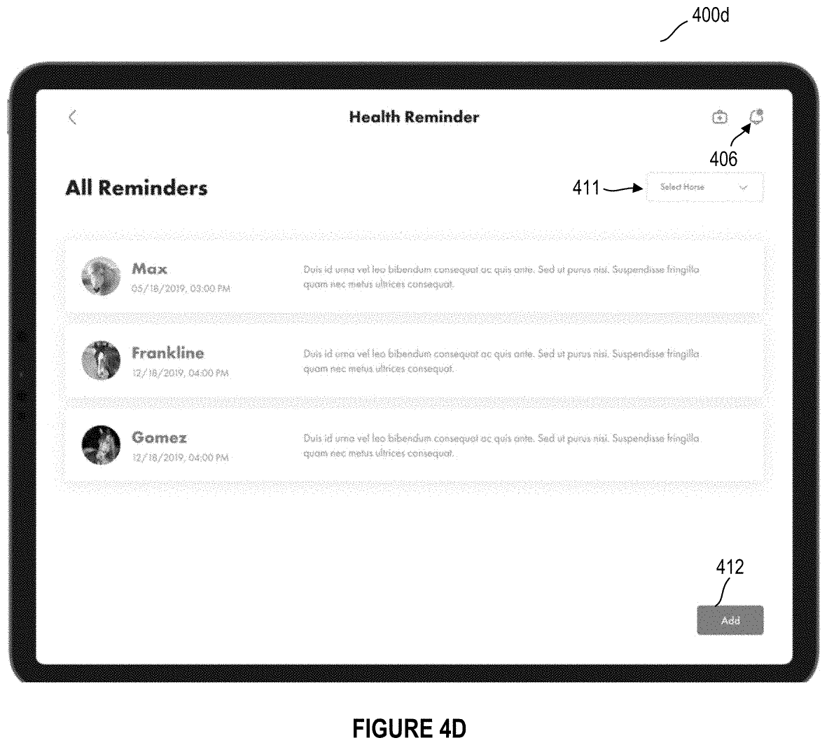

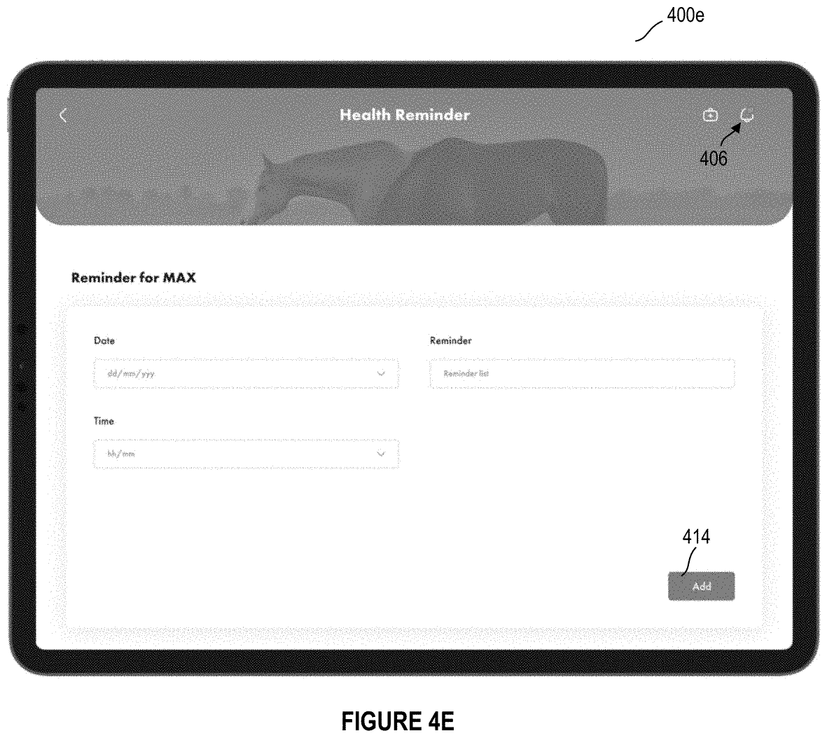

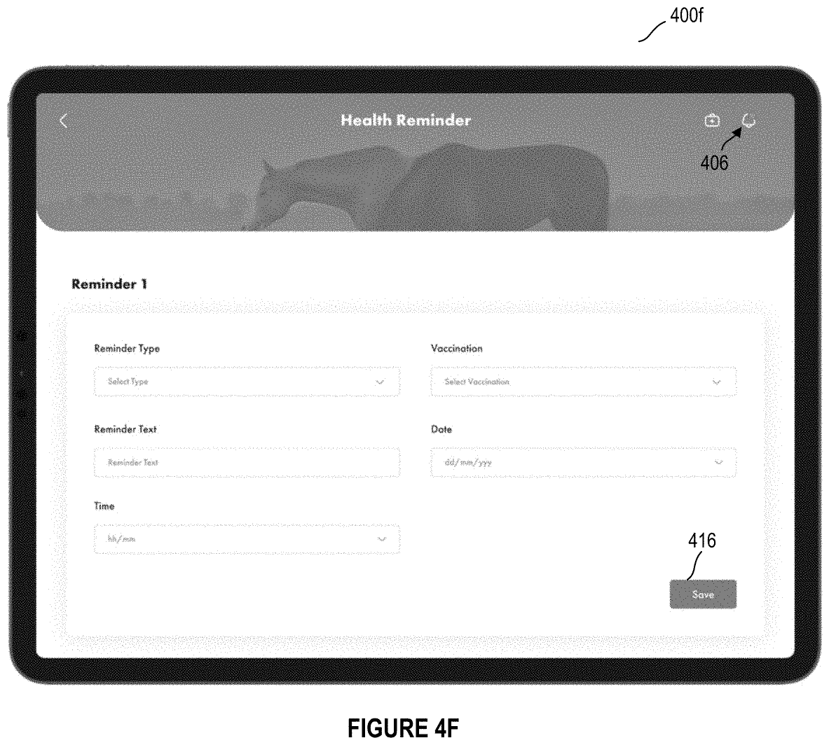

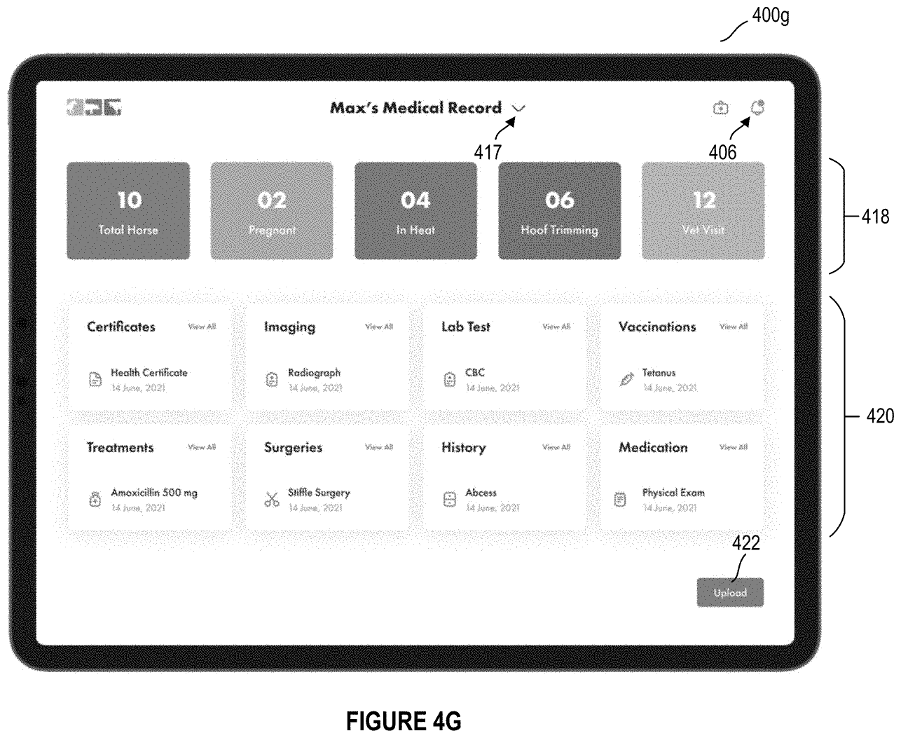

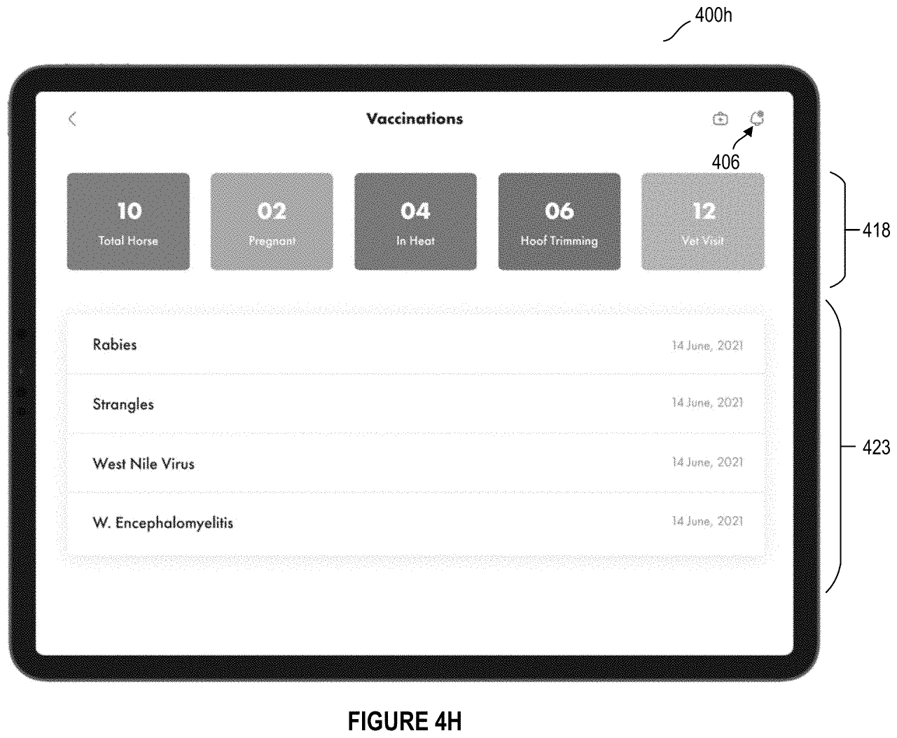

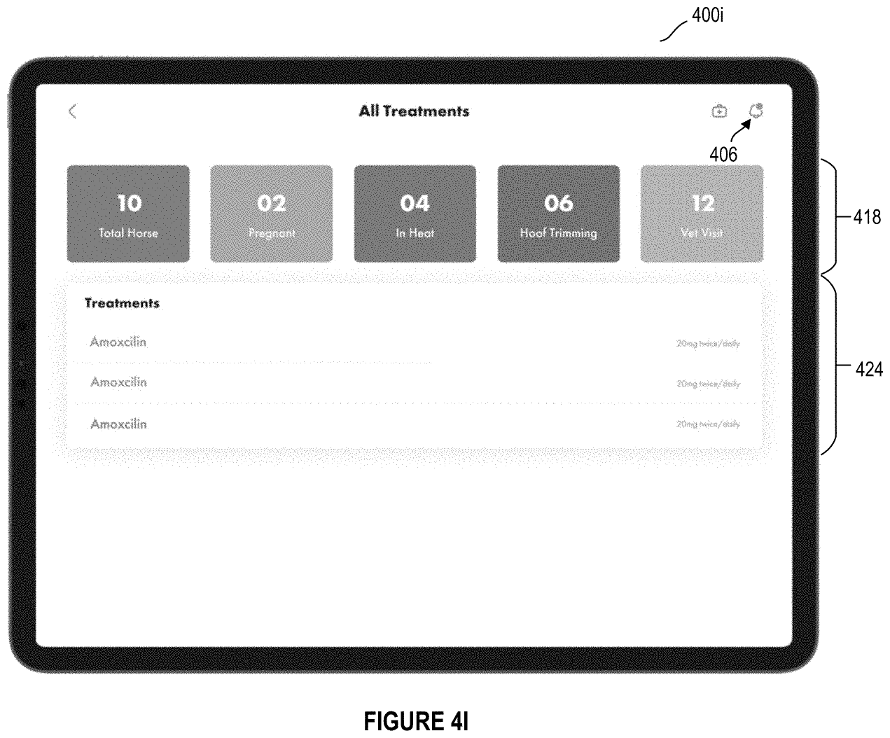

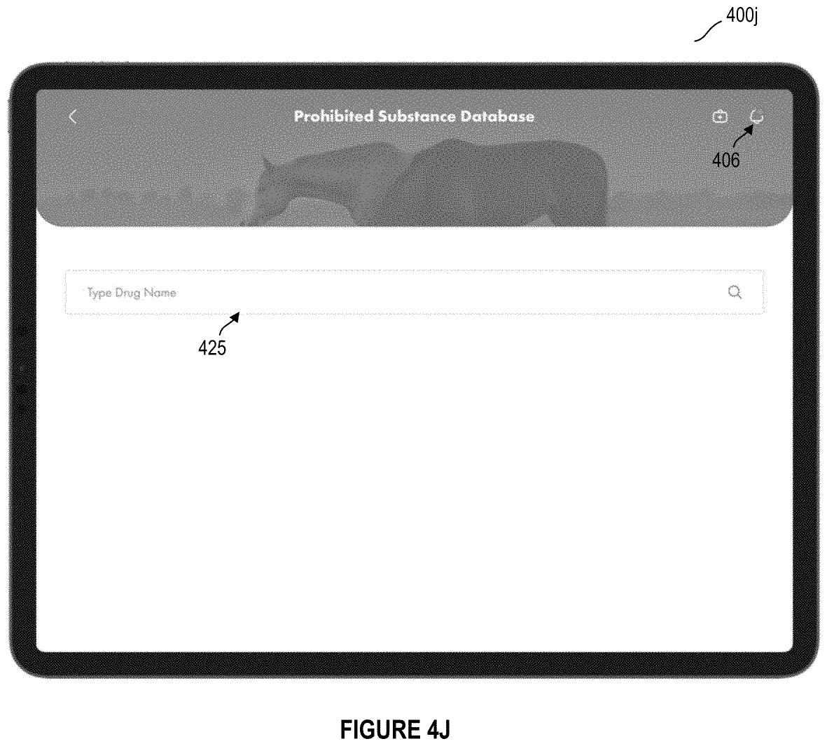

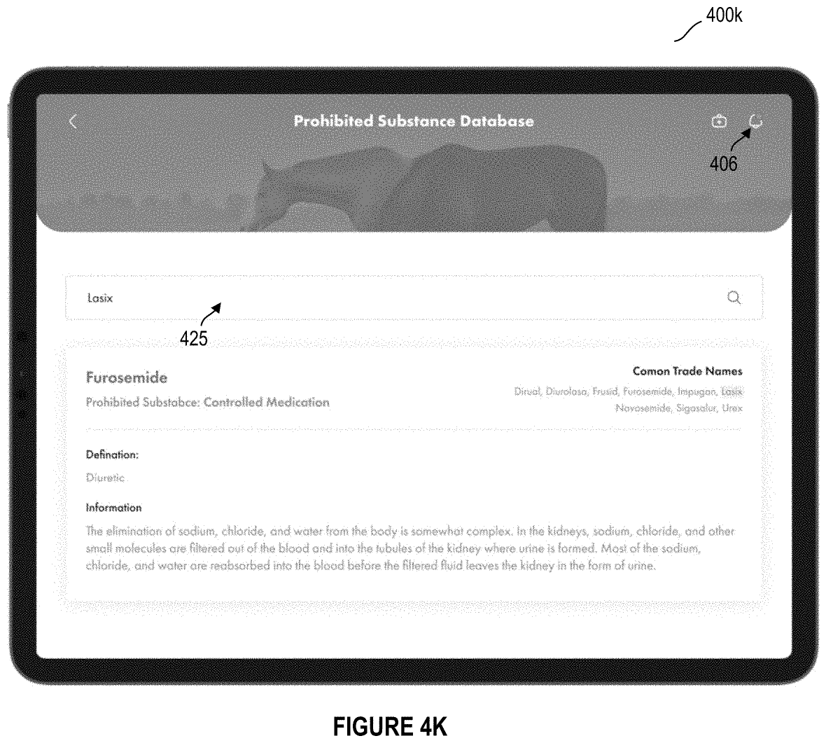

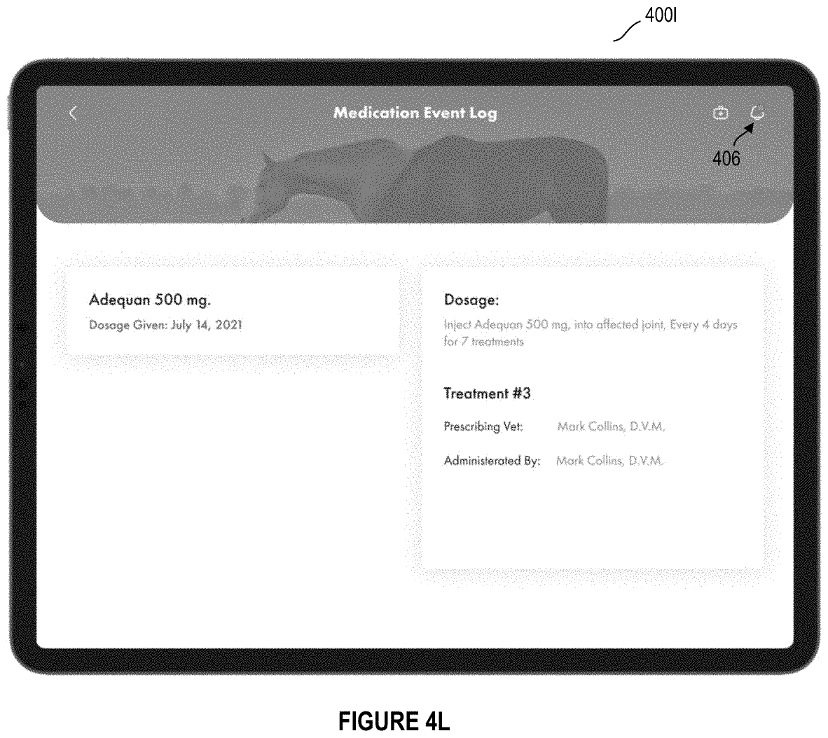

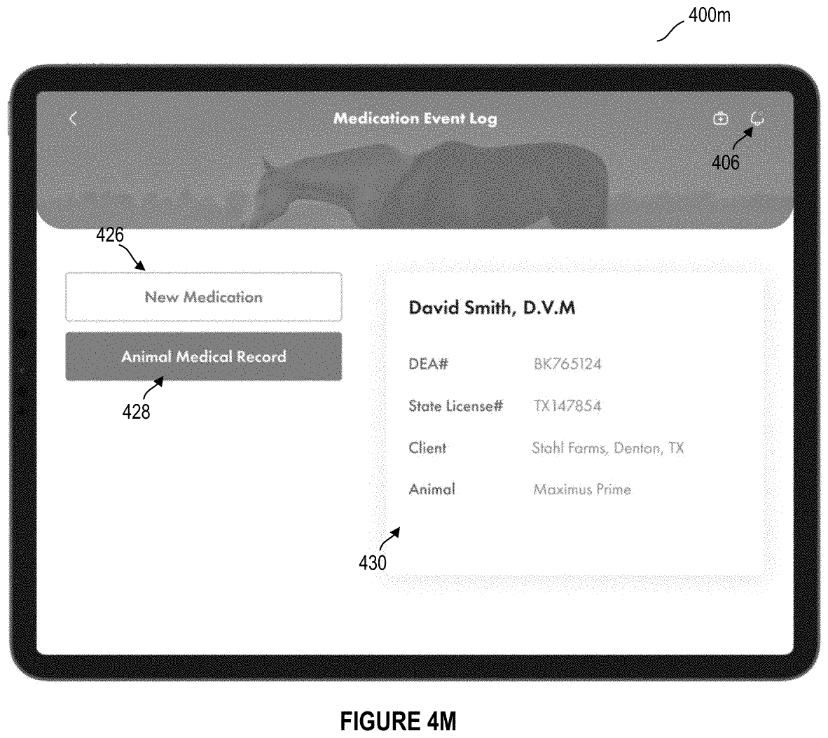

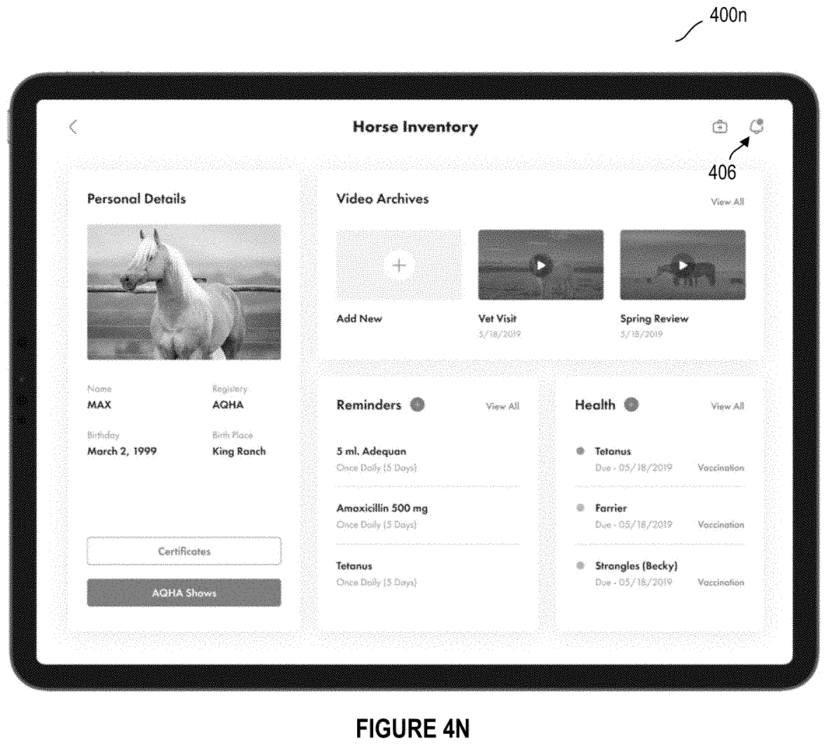

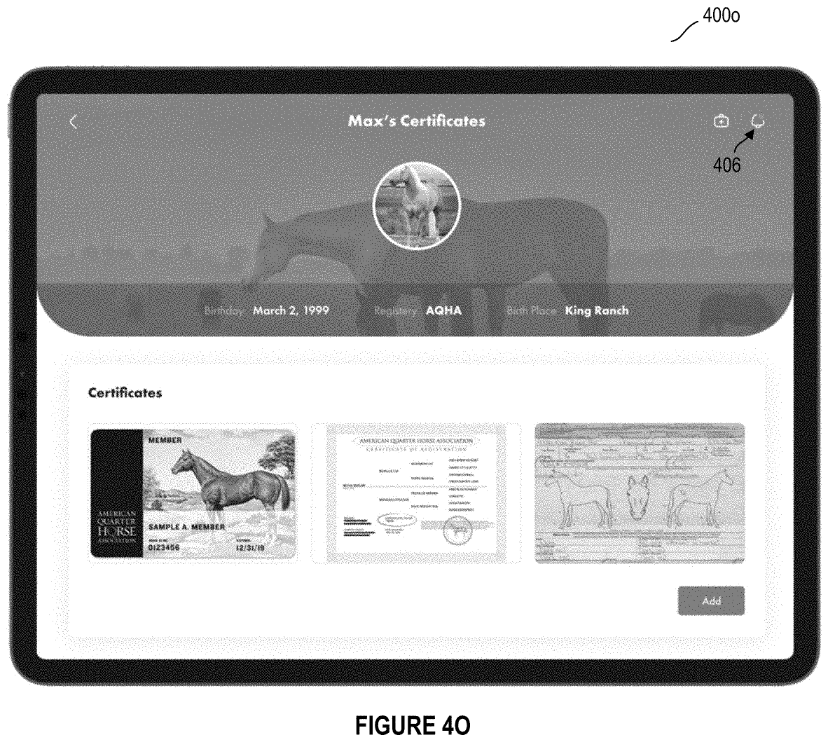

FIGS. 4A-4O illustrate example user interfaces 400 that may be generated for managing animal health, according to certain embodiments. For example, user interfaces 400 may be user interfaces that are generated by animal management application 210 stored on user devices 102. Although labeled user interface 400a, 400b, 400c, and so on, the user interfaces may be referred to generally as user interfaces 400.

Although particular user interfaces 400 are illustrated and described, this disclosure contemplates animal management application 210 generating these and/or other user interfaces. Although user interfaces 400 are illustrated and described as being arranged in a particular way, including particular content, or the like, user interfaces 400 and the particular arrangement and content of user interfaces 400 are for example purposes only.

Although primarily described in connection with an implementation for managing horses, embodiments of this disclosure may be used to manage any suitable type of animal, including, for example, pigs, cows, chickens, horses, sheep, or any other suitable type of animal.

As illustrated in FIG. 4A, user interface 400a includes an account identifier 402, which may identify one or more of an overall account (e.g., a corporate account associated with a farm), a particular user 110, or the like.

User interface 400a may present a dashboard of menu options for managing animal health. For example, user interface 400a may include multiple selectable links 404a-404g (referred to generally as selectable links 404) that correspond to an associated feature and data set for managing animal health. Links 404 may operate as portals to particular data and feature sets associated with managing animal health.

In the illustrated example, a link 404a provides a link to the dashboard (e.g., user interface 400a). Link 404b provides a link to an away care user interface for providing data related to caring for the inventory of animals when the owner/operator is away. Link 404c provides a link to a veterinarian chat user interface for engaging in an electronic interaction with a veterinarian. Link 404d provides a link to a health reminders user interface for the animals in the animal inventory. Link 404e provides a link to a breeding record user interface for some or all of the animals in the animal inventory. Link 404f provides a link to a medical record user interface for the animals in the animal inventory. Link 404f provides a link to a racing user interface for managing data associated with animals (e.g., horses) that may be used for racing.

Selecting links 404 may cause animal management application 210 to access suitable digital animal records 112 stored on storage unit 106 and display, in a suitable user interface, information corresponding to the selected link 404. For example, in response to a user request, animal management application 210 may interact with animal management logic 310 to search digital animal records 112 stored on storage unit 106 to provide for display in one or more additional user interfaces 400 information responsive to the user request.

User interface 400a includes a notification 406 that indicates the availability of one or more reminders, such as reminders that may be further explained in the detailed health reminder page linked by link 404d. As will be seen throughout FIGS. 4B-4O, notification 406 may be persistently shown through some or all of user interfaces 400, so that the associated reminders may be quickly accessed from any of a variety of user interfaces 400.

As illustrated in FIG. 4B, user interface 400b includes an away care user interface. The away care user interface may be particularly useful when the animal owner (or an employee or other person associated with the owner who cares for the animals) is away and arranges for another entity (person or company, for example) to complete various tasks associated with raising the horses.

In the illustrated example, user interface 400b includes a date selector/indicator 408, which can be used to select away care tasks for a particular date. In this example, the date Wednesday, 17 Apr., 2019, is selected, and aware care tasks for one or more horses in the horse inventory that are scheduled for that date are shown. In particular, in the illustrated example and for the date specified, user interface 2oob shows aware care tasks for three horses, Bella, Max, and Cobia. Taking Bella as an example, the listed away care tasks include a morning feeding (AM feeding), the feeding food type and amount (e.g., three scoops of Nutrena Performance feed), and replacing Bella's water. Additionally, in the illustrated example, user interface 400b includes a photo for each horse and a time for performing the listed away care tasks (e.g., 6:30 AM). Furthermore, in the illustrated example, user interface 400b identifies, for each horse, the entity responsible for performing the away care tasks. In this particular example, user interface 400b indicates that an individual named John Cooper is responsible for the away care tasks for all three listed horses.

As illustrated in FIG. 4C, user interface 400c includes a veterinarian video chat (Vet Video Chat) feature. User interface 400c may allow an owner or other individual caring for a horse to engage in a video chat with a veterinarian without requiring the veterinarian to visit the farm or the owner (or other caretaker) to bring the animal to the veterinarian for evaluation. This may be particularly useful for initial consultations, issues that arise during non-business hours, or in other suitable scenarios. Furthermore, the veterinarian may be able to view certain portions of the horse to possibly provide an evaluation over the video chat.

User interface 400c includes a video consultation window 410 through which the video of the veterinarian may be displayed. In addition to including the video consultation window, in the illustrated example, user interface 400c includes a history window that lists recent veterinarian events for that horse and the associated date and veterinarian (e.g., Procedure, Jul. 7, 2019, by Dr. West Wood), and includes a prescription window that lists the prescriptions for the horse (e.g., Amoxicillin, Pen-B, and Phenylbutazone).

As illustrated in FIG. 4D, user interface 400d includes health reminders for the horses in the horse inventor. In certain embodiments, user interface 400d is displayed in response to a user selecting notification 406. The health reminder may be particularly useful for quickly accessing the health reminder events associated with horses in the horse inventory for which active health reminders exist.

In the current view of user interface 400d, health reminders for all horses that have an active reminder (All Reminders) are displayed. In this example, active health reminders exists for horses named Max, Frankline, and Gomez. A particular date for the health reminder also is listed in association with each horse. User interface 400d may include a horse-selector dropdown menu 411 for selecting a particular horse for which to view health reminders. In such an example, if a particular horse is selected (e.g., Max), then rather than showing all reminders, user interface 400d may be updated to show health reminders for the particular horse (e.g., Max), along with potentially additional detail regarding those reminders. User interface 400d also includes an Add button 412, which may allow a user to add a new health reminder.

As illustrated in FIG. 4E, user interface 400e may facilitate adding a health reminder. For example, animal management application 210 may display user interface 400e in response to receiving a user selection of Add button 412 from user interface flood, including potentially from a detailed health reminder version of user interface 400d for a particular horse (e.g., Max). As another example, animal management application 210 may display user interface 400e in response to receiving a user selection of an add reminder button from a detailed view of information for a particular horse (e.g., Max), an example of which is shown in FIG. 4N, described below.

In the illustrated example, user interface 400e includes a horse identifier (e.g., “Reminder for Max”), a date field for specifying a date for the reminder, a time field for specifying a time for the reminder, and a reminder field for selecting a type of the reminder from a predefined list of reminders. Add button 414 may be selected to cause animal management application 210 to add the reminder, which may include communicating the reminder to animal management logic 310 for updating digital animal records 112.

As illustrated in FIG. 4F, user interface 400f also may facilitate adding a health reminder, which in this case may be specific to adding a vaccination reminder. For example, animal management application 210 may display user interface 400e in response to receiving a user selection of Add button 412 from user interface flood, including potentially from a detailed health reminder version of user interface 400d for a particular horse (e.g., Max). As another example, animal management application 210 may display user interface 400e in response to receiving a user selection of an add reminder button from a detailed view of information for a particular horse (e.g., Max), an example of which is shown in FIG. 4N, described below.

In the illustrated example, user interface 400e includes a reminder type field for selecting a type of the reminder from a predefined list, a reminder text field for entering freeform text associated with the reminder, a time field for specifying a time for the reminder, a vaccination field for selecting from a predefined list a particular vaccination associated with the reminder, and a date field for specifying a date for the reminder. Save button 416 may be selected to cause animal management application 210 to add the reminder, which may include communicating the reminder to animal management logic 310 for updating digital animal records 112.

As illustrated in FIG. 4G, user interface 400g includes a digital medical record for a selected horse of the horse inventory. For example, from user interface 400a of FIG. 4A, a user may select link 404f to user interface 400g for some or all of the horses in the horse inventory. Selecting link 404f from user interface 400a may cause animal management application 210 to access via storage unit 106 animal health data for a particular subdivision of the farm and display user interface 400g that provides digital medical records. In this example, user interface 400g is the medical record page for a particular horse (Max, as indicated by the “Max's Medical Record” label). Adjacent to the “Max's Medical Record” label, a dropdown arrow 417 may be selected to choose the medical records for a different horse or possibly a summary for all horses in the horse inventor (or a particular subdivision thereof). For example, in response to selecting dropdown arrow 417, animal management application 210 may display a list (e.g., in a dropdown list) of available horses (or subdivisions thereof, including potentially all) from which a desired selection can be made and in response to which animal management application 210 may update user interface 400g to display data associated with the selection. If appropriate, to update user interface 400g according to a selection, animal management application 210 may communicate with animal management logic 310 to retrieve responsive data for display in the updated user interface 400g.

In the illustrated example, user interface 400g includes a summary banner 418 and health information blocks 420, as limited by the selection made using dropdown arrow 417.

For quick reference, summary banner 418 includes, in various categories, a summary of the number of horses that fit into the identified categories. In this example, the categories include the total number of horses (e.g., 10), the number of pregnant horses (e.g., 2), the number of horses in heat (e.g., 4), the number of horses that need hoof trimming (e.g., 6), and the number of horses that are due for a visit by the veterinarian (e.g., 12, which could be greater than the total number of horses if, for example, a particular horse needs to see the veterinarian for multiple different reasons).

In the illustrated example, user interface 400g includes information blocks 420 for categories of health information for the horse selected using dropdown arrow 417. In this example, the information blocks 420 for categories of health information include Certificates, Imaging, Lab Test, Vaccinations, Treatments, Surgeries, History, and Medication. Although these particular information blocks 420 are shown, this disclosure contemplates user interface 400g including any suitable information blocks for any suitable health information.

Information blocks 420 may include an example detailed entry for the health information associated with that information block 420. For example, the Certificates information block 420 includes a listing for a health certification dated Jun. 14, 2021, which could be the most recent health certificate for Max. As another example, the Imaging information block 420 includes a listing for a radiography data (e.g., an X-ray) dated Jun. 14, 2021, which could be the most recent radiography data for Max. As another example, the Lab Test information block 420 includes a listing for a lab test (e.g., a complete blood count, or CBC lab test) dated June 14, 2021, which could be the most recent lab test for Max. As another example, the Vaccinations information block 420 includes a listing for a vaccination (e.g., Tetanus) dated June 14, 2021, which could be the most recent vaccination for Max. As another example, the Treatments information block 420 includes a listing for a drug treatment (e.g., Amoxicillin at 500 mg) dated Jun. 14, 2021, which could be the most recent health treatment for Max. As another example, the Surgeries information block 420 includes a listing for a surgery (Stifle Surgery) that occurred on Jun. 14, 2021, which could be the most recent surgery for Max. As another example, the History information block 420 includes a listing for a health event (e.g., an Abscess) dated Jun. 14, 2021, which could be the most recent health event (e.g., illness) for Max. As another example, the Medication information block 420 includes a listing for a physical examination performed on Jun. 14, 2021, which could be the most recent health examination (and potential administration/prescription of medication) for Max.

In certain embodiments, selecting the example detailed entry for the health information associated with a particular information block 420 may cause certain further details associated with that example detailed entry to be accessed and displayed. For example, in response to selection of the icon to the left of the health certification dated Jun. 14, 2021, entry, animal management application 210 may access (potentially with a request to animal management logic 312 for retrieval from digital animal records 112) and display an image of the certificate associated with the health certification dated Jun. 14, 2021. Similar operations may occur in response to selection of the icon adjacent to the example detailed entry for other information blocks 420.

In this example, each information block 420 includes a “View All” link, which, when selected, may cause animal management application 210 to access (potentially with a request to animal management logic 312 for retrieval from digital animal records 112) and display all data (potentially limited to a designated time frame or number of entries) for the health information category associated with that information block 420. For example, in response to selection of the “View All” link for the Certificates information block 420, animal management application 210 may access and display all certificates for Max. Similar operations may occur in response to selection of the “View All” link for other information blocks 420.

An Upload button 422 may be selected to allow additional information to be added to the medical record for Max. Adding additional information to Max's medical record may cause animal management application 210 to communicate the added information to animal management logic 312 for addition to digital animal records 112.

As illustrated in FIG. 4H, user interface 400h includes a vaccine list 423 of vaccinations that have been administered to a particular horse (e.g., Max), along with a date on which the vaccine was administered. In the illustrated example, user interface 400h again includes summary banner 418.

As illustrated in FIG. 4I, user interface 400i includes a treatment list 424 of treatments (e.g., medications) that have been and/or are being administered to a particular horse (e.g., Max), along with instructions (e.g., dosage and frequency) for administering the treatment. In the illustrated example, user interface 400h again includes summary banner 418.

As illustrated in FIG. 4J, user interface 400j provides access to a prohibited substance database and includes a search bar 425 for entering a drug name to determine whether that drug appears in the prohibited substance database. In certain embodiments, user interface 400j is a part of the user interfaces 400 made available by selecting link accessed using link 404g (the Racing link) from the dashboard of user interface 400a. In response to entry of a drug name, animal management application 210 may access a prohibited substance database for a determination of whether the entered drug is identified as a prohibited drug and, in response, display a search result (e.g., not listed or listed). The prohibited drug list may be maintained by a suitable racing authority (e.g., the American Quarter Horse Association (AQHA)), a government, or another suitable entity.

In certain embodiments, the prohibited substance database could be provided by storage unit 106 (e.g., as part of digital animal records 112 or separately), but this disclosure contemplates the prohibited substance database being provided and managed by any suitable entity. For example, the prohibited substance database could be managed by a third party, such as a racing authority (e.g., the AQHA), a government entity, or another suitable entity. As another example, processing system 104 (e.g., animal management logic 312) may update a prohibited substance database on storage unit 106 by accessing a prohibited substance database maintained by the third party and updating prohibited substance database on storage unit 106 with the updated information.

As illustrated in FIG. 4K, user interface 400k provides an example user interface that may be displayed if a drug entered in search bar 425 of user interface 400k (and shown again in user interface 400k) is located in the prohibited substance database. In this example, the entered drug name is Lasix, and the returned result includes the formal name for the drug (Furosemide), common trade names for the drug (e.g., including the drug name entered in search bar 425 in this example), a status of the drug (e.g., Controlled Medication), and additional details related to the substance.

As illustrated in FIG. 4L, user interface 4001 may provide a medication event log for a particular horse (e.g., Max). In this example, the medication event includes the medicine and dosage prescribed (e.g., Adequan at 500 mg.), a date of the prescription (Jul. 14, 2021), additional dosage details (e.g., “Inject Adequan 500 mg, into affected joint, every 4 days for 7 treatments”), and the prescribing veterinarian (Mark Collins, D.V.M.) and administering individual (Mark Collins, D.V.M.).

As illustrated in FIG. 4M, user interface 400m may provide another interface of the medication event log for a particular horse (e.g., Maximus Prime, for which Max may be the short name). In this example, user interface _{d}oom includes a “New Medication” button 426 for accessing an interface for entering a new medication prescribed to the particular horse (e.g., Max) and an “Animal Medical Record” button 428 for causing animal management application 210 to display the medical record (e.g., user interface 400g of FIG. 4G) for the particular horse (e.g., Max). User interface 400m also may include an information block 430 that includes details regarding the prescribing veterinarian (David Smith, D.V.M.), a federal registration number for the veterinarian (e.g., Drug Enforcement Agency number (DEA#), a state registration number (e.g., state license number), a client identifier (e.g., the farm at which the horse is being raised), and an animal identifier (e.g., Maximus Prime, or Max).

As illustrated in FIG. 4N, user interface 400n provides a view of detailed information for a particular horse in the horse inventory. User interface 400n may include personal details of the particular horse, such as a name (e.g., Max), an agency with which the horse is registered (if applicable) (e.g., AQHA), a birthday, and a birthplace. User interface 400n may include additional personal details of the particular horse, such as a link to any associated certificates (see, e.g., FIG. 4O, described below) and a link to shows for the entity with which the horse is registered (e.g., AQHA Shows). User interface 400n may include one or more videos of the particular horse, along with a link for adding further videos. For example, the videos for the particular horse may include videos of one or more veterinarian visits, a periodic review, and the like. User interface 400n may include a reminders section that lists one or more reminders for the particular horse. Example reminders may include scheduled medications, scheduled vaccinations, scheduled veterinary visits, and the like. User interface 400n may include a health section that lists one or more reminders for administering medication (e.g., vaccinations), along with a due date.

In this example, each of the video archives, reminders, and health sections includes a “View All” link, which, when selected, may cause animal management application 210 to access (potentially with a request to animal management logic 312 for retrieval from digital animal records 112) and display all data (potentially limited to a designated time frame or number of entries) for the videos, reminders, and health sections. For example, in response to selection of the “View All” link for the Video Archives section, animal management application 210 may access and display all videos (or at least a link (possibly with a thumbnail image) to play the videos) for Max. Similar operations may occur in response to selection of the “View All” link for the reminders and health sections. Furthermore, the reminders and health sections may include a link (e.g., a plus sign) to add a reminder entry or health entry, respectively.

As illustrated in FIG. 4O, user interface 400o may include one or more certificates for a particular horse, as well as a link for adding further certificates for the particular horse. An example certificate for the particular horse may include a membership registration card for a particular agency (e.g., the AQHA), a certificate of registration with a particular agency (e.g., the AQHA), an inspection form (e.g., an Equine Inspection Anemia Test Form, such as a so-called Coggins report), or other suitable documents. In certain embodiments, user interface 400o for a particular horse may be accessed by selecting the certificates link on user interface 400n for the particular horse.

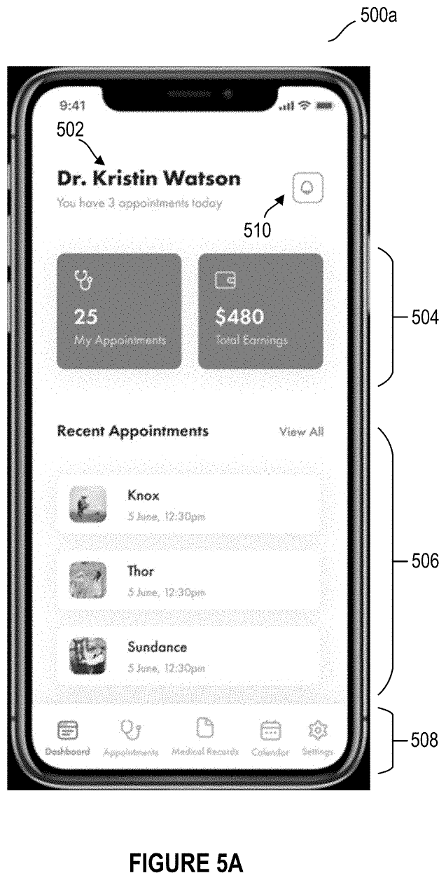

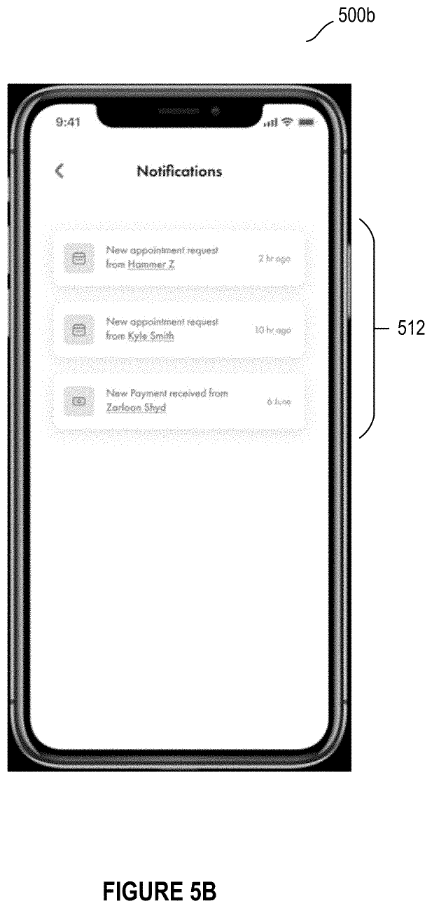

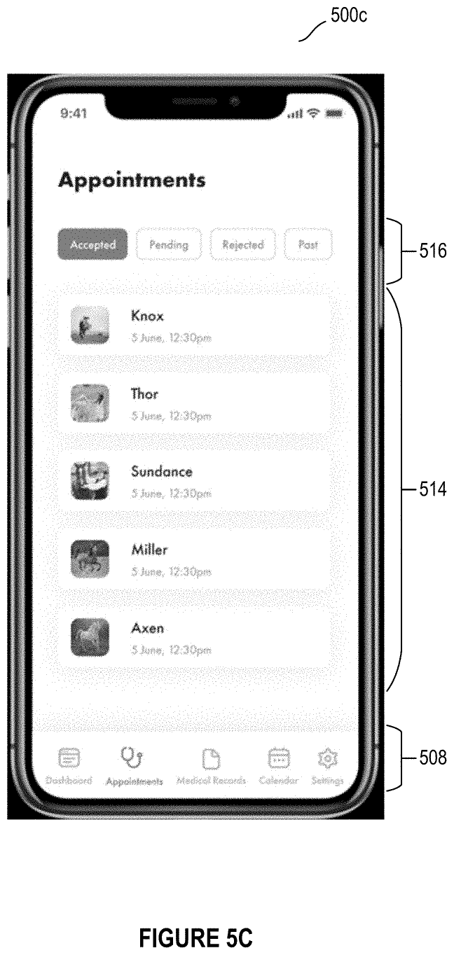

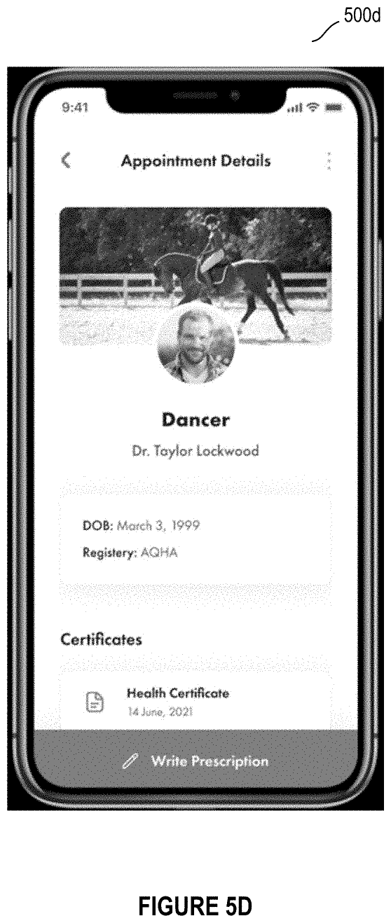

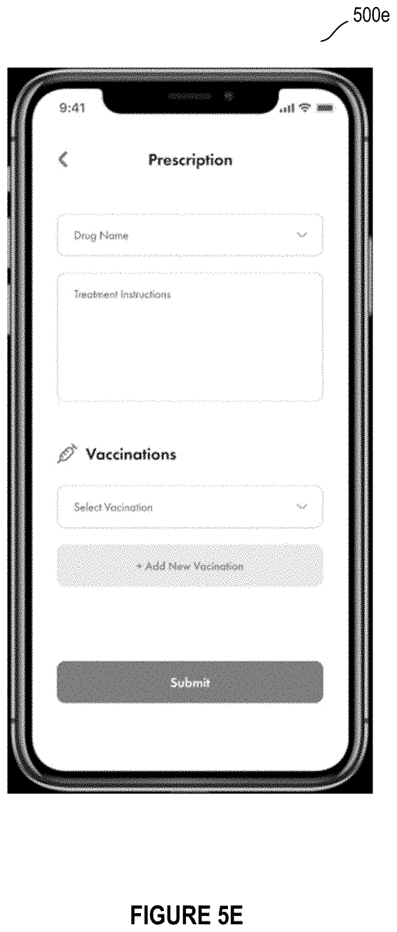

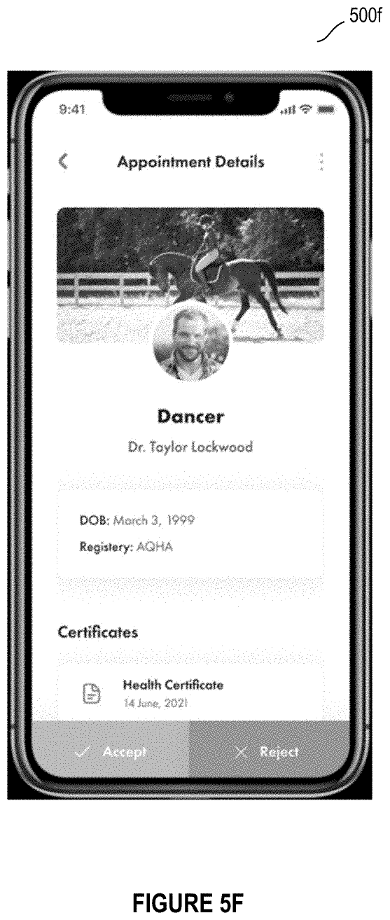

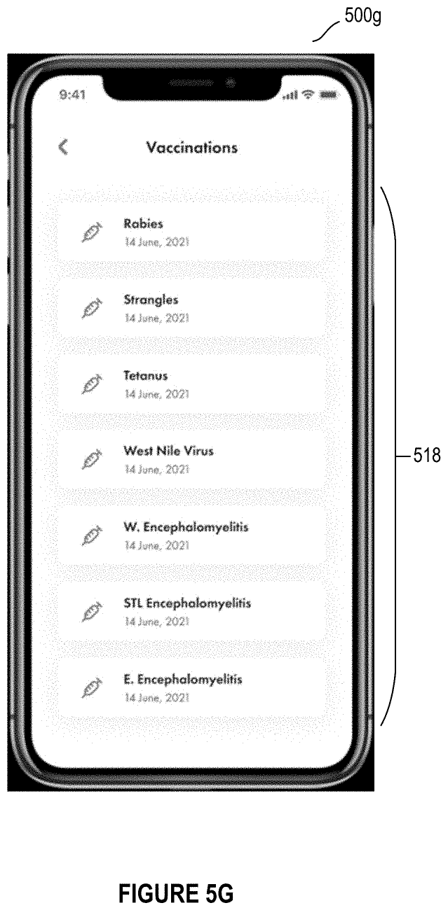

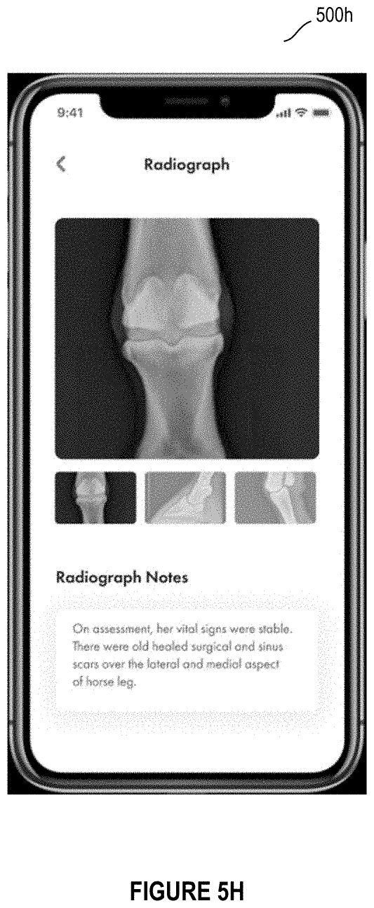

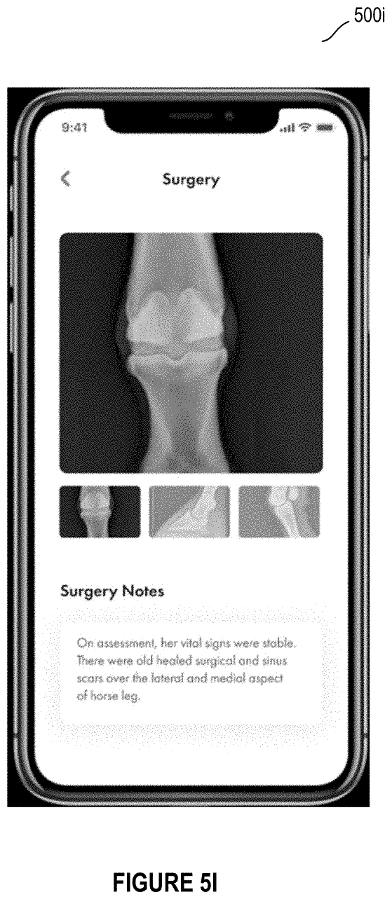

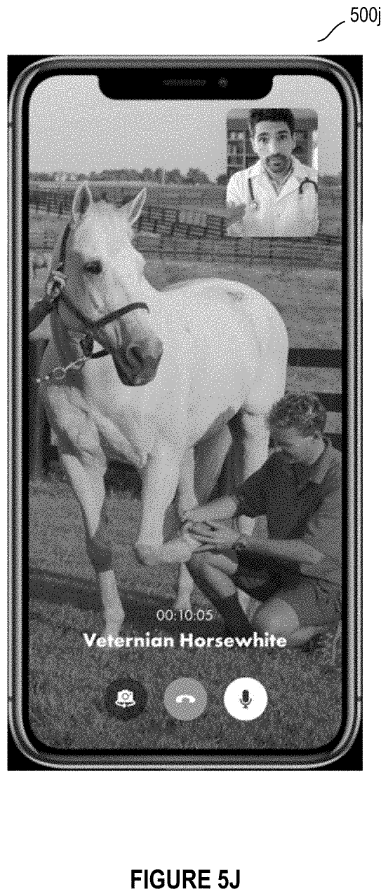

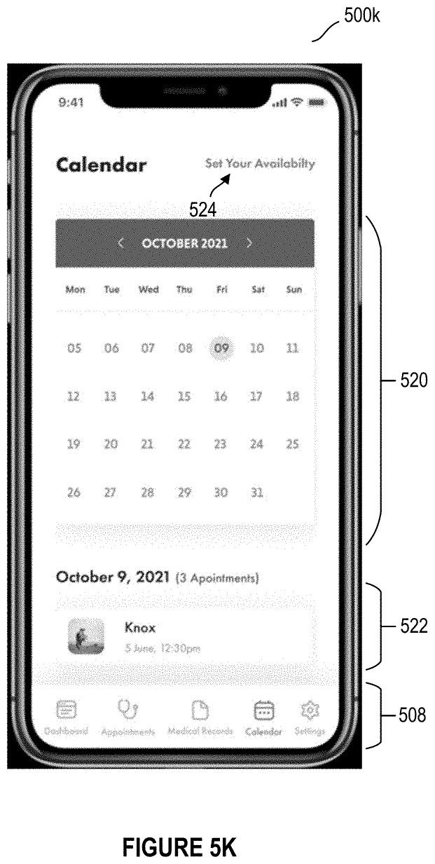

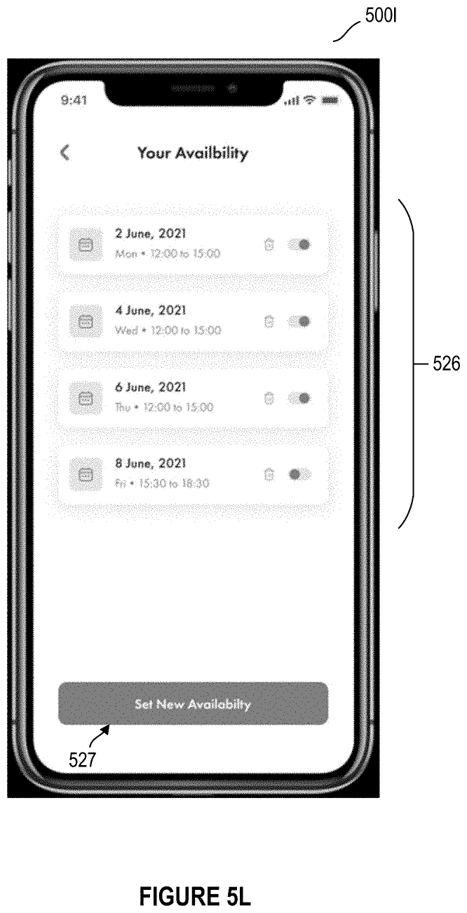

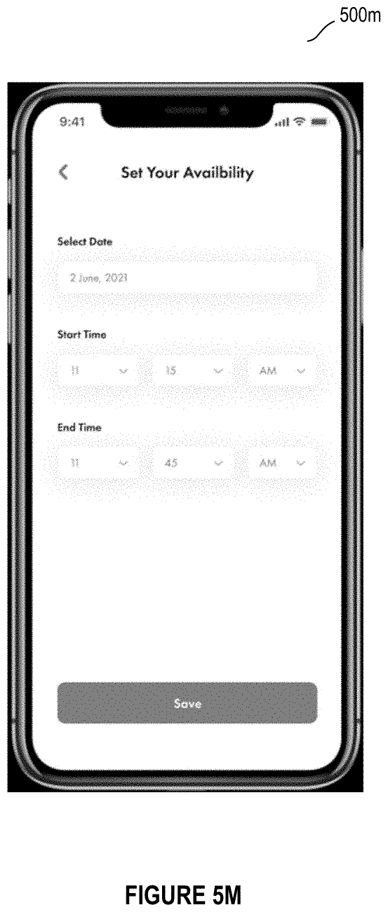

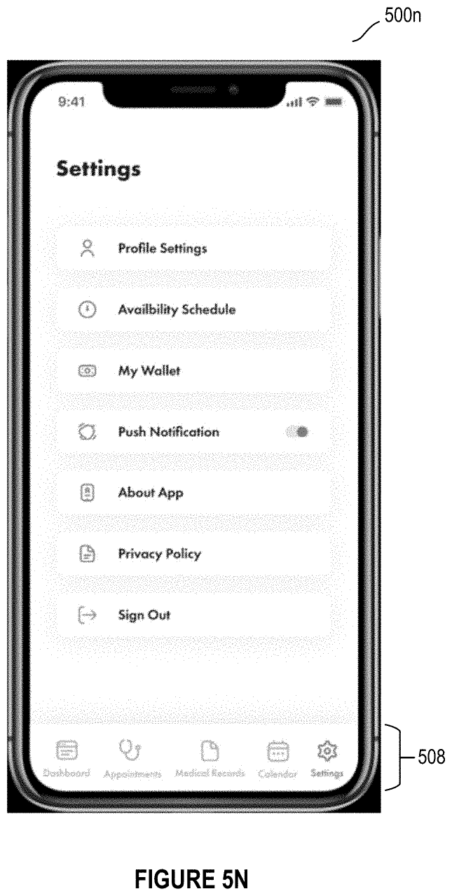

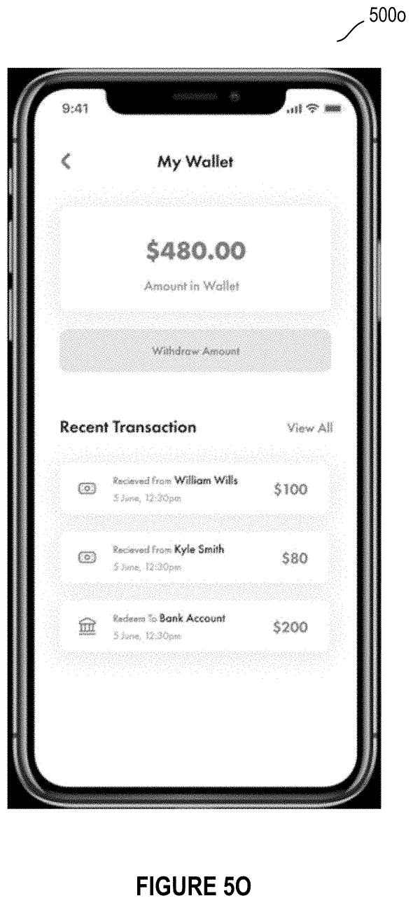

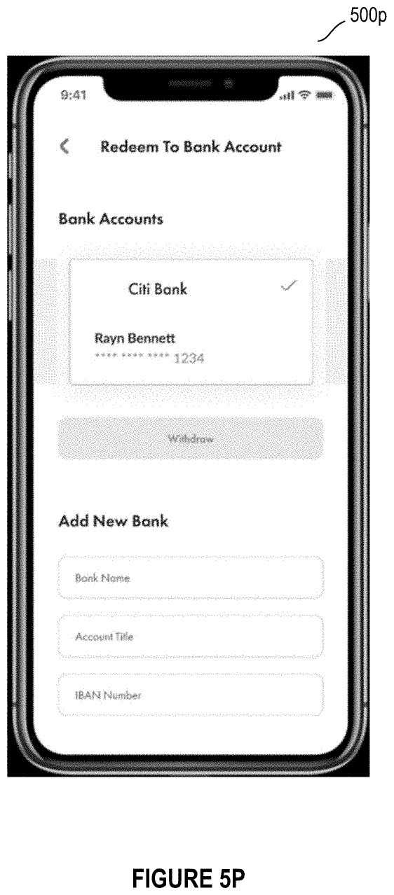

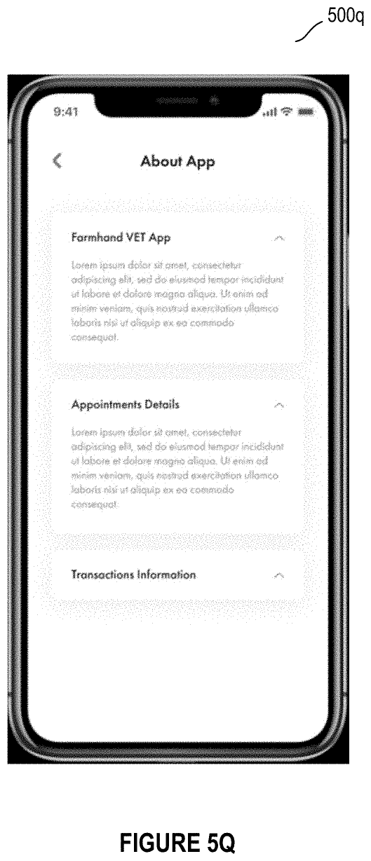

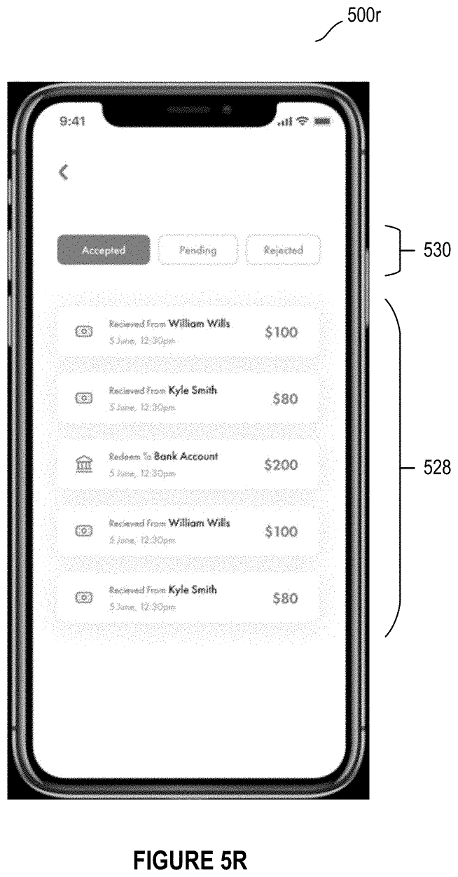

FIGS. 5A-5R illustrate example user interfaces 500 that may be generated for providing certain features for a veterinarian, according to certain embodiments. For example, user interfaces 500 may be user interfaces that are generated by animal management application 210 stored on user devices 102. Although labeled user interface 500a, 500b, 500c, and so on, the user interfaces may be referred to generally as user interfaces 500.

Although particular user interfaces 500 are illustrated and described, this disclosure contemplates animal management application 210 generating these and/or other user interfaces. Although user interfaces 500 are illustrated and described as being arranged in a particular way, including particular content, or the like, user interfaces 500 and the particular arrangement and content of user interfaces 500 are for example purposes only.

Although primarily described in connection with an implementation for managing horses, embodiments of this disclosure may be used to manage any suitable type of animal, including, for example, pigs, cows, chickens, horses, sheep, or any other suitable type of animal.

As illustrated in FIG. 5A, user interface 500a includes an account identifier 502, which may identify one or more of an overall account (e.g., a corporate account associated with a veterinary clinic), a particular veterinarian (e.g., a particular user no), or the like. For purposes of describing FIGS. 5A-5R, it will be assumed that the user no is a particular veterinarian (e.g., Dr. Kristin Watson), as indicated at account identifier 502.

User interface 500a may present a dashboard of certain overview information for the veterinarian, examples of which are described below.

For example, adjacent to account identifier 502, user interface 500a may include an indication of the number of appointments for the current day (e.g., “You have 3 appointments today”).

As another example, user interface 500a may include a summary banner 504 includes multiple boxes with different categories of information. In the illustrated example, summary banner 504 includes a box that indicates the total number of appointments (“My Appointments,” indicating 25) and a box that indicates the total earnings (“Total Earnings,” indicating $480). In certain embodiments, the boxes in summary banner 504 may operate as links that, when selected, cause animal management application 210 to display additional details regarding the contents of the selected box (e.g., additional appointment detail or additional earnings detail).

As another example, user interface 500a includes an appointments section 506 that lists recent/upcoming appointments, along with the horse to be seen and the date and time of the appointment. In this example, appointments section 506 includes a “View All” link, which, when selected, may cause animal management application 210 to access (potentially with a request to animal management logic 312 for retrieval from digital animal records 112) and display all data (potentially limited to a designated time frame or number of entries) for all appointments for the veterinarian. Additionally or alternatively, in certain embodiments, the box for “My Appointments” in summary banner 504 may operate as a link to view details of some or all of the appointments.

As another example, user interface 500a may include a dock 508 that includes links to particular feature sections provided by animal management application 210. In the illustrated example, dock 508 includes feature sections for Dashboard, Appointments, Medical Records, Calendar, and Settings. In certain embodiments, animal management application 210 may allow the feature sets available in dock 508 to be configured by a user, using the settings section for example. The links in dock 508 may operate as portals to particular data and feature sets associated with veterinarian features for managing animal health. Selecting links from dock 508 may cause animal management application 210 to access suitable digital animal records 112 stored on storage unit 106 and display, in a suitable user interface, information corresponding to the selected link. For example, in response to a user request, animal management application 210 may interact with animal management logic 312 to search digital animal records 112 stored on storage unit 106 to provide for display in one or more additional user interfaces 100 information responsive to the user request.

The Dashboard link may lead to user interface 500a, the Appointments link may lead to user interface 500c (described below), the Medical Records link may lead to a medical records user interface (described below), the Calendar link may lead to user interface 500k (described below), and the Settings link may lead to user interface 500n (described below).

User interface 500a includes a notification 510 that indicates the availability of one or more notifications. For example, selecting notification 510 may lead to user interface 500b, described below. As will be seen throughout FIGS. 5B-5R, notification 510 may be persistently shown through some or all of interfaces 500, so that the associated notifications may be quickly accessed from any of a variety of user interfaces 500.

As illustrated in FIG. 5B, user interface 500b may provide an overview of notifications for the veterinarian. As described above, in certain embodiments, user interface 500b may be reached by selecting notification 510. In the illustrated example, user interface 500b may include a notifications list 512 that includes any suitable notifications for the veterinarian. In this example, notifications list 512 includes two notifications for requests for appointments and one notification that a payment was received. For each notification in notifications list 512, user interface 500b may include a time associated with the notification, which could be expressed as an age (e.g., 2 hr ago or 10 hr ago) or a date (e.g., 6 June). In certain embodiments, the notifications in notification list 512 include a selectable icon (e.g., to the left) that operates as a link to one or more further user interfaces for viewing additional details related to the notification. In the illustrated example, the first two notifications may lead to additional details related to the appointment request, while the third notification may lead to additional details related to the submitted payment.

As illustrated in FIG. 5C, user interface 500c may include an appointment list 514, which lists zero or more appointments. As described above, in certain embodiments, user interface 500c may be reached by selecting the appointments link from dock 508, by selecting the My Appointments box in summary banner 504 of user interface 500a (e.g., the Dashboard), or in any other suitable manner. The listed appointments may include a horse that was/is/would be the subject of the appointment, along with a date for the appointment. In certain embodiments, a picture, possibly of the subject horse, may be displayed adjacent to the listed appointment.

User interface 500c may include a filter banner 516, which may provide a number of options for filtering which appointments are shown in appointments list 514. In the illustrated example, the filtering options of filter banner 516 include Accepted (for displaying in appointments list 514 appointments that have been accepted), Pending (for displaying in appointments list 514 appointments that are pending (e.g., have been requested but have not been accepted or rejected)), Rejected (for displaying in appointments list 514 appointments that have been rejected), and Past (for displaying appointments that occurred in the past).

As illustrated in FIG. 5D, user interface good may provide details for a particular horse (e.g., Dancer). In certain embodiments, user interface good may be considered part of the digital medical record (e.g., is an overview medical record screen) for a particular horse (e.g., for the horse associated with the appointment). The listed details may include the horse's name (e.g., Dancer), the horse's veterinarian (Dr. Taylor Lockwood, which might be, for example, the horse's typical veterinarian and/or the veterinarian with which the appointment is associated), personal details associated with the identified horse (e.g., date of birth and/or registry), links to one or more certificates (e.g., a health certificate, including, possibly, a date of the most recent health certificate), or other information. Although not visible in the illustrated example of user interface 500e, scrolling down may reveal other links to other information about Dancer. For example, additional information (and potentially links to further details) for medical records, vaccinations, breeding details, health history, treatment history, and/or any other suitable information may be provided.

In certain embodiments, user interface good includes a Write Prescription link for the veterinarian to write a prescription directly from the application (e.g., animal management application 210). In certain embodiments, the Write Prescription link may cause animal management application 210 to display user interface 500e, described below.

As illustrated in FIG. 5E, user interface 500e provides an interface for adding a prescription. In certain embodiments, user interface 500e may be reached by selecting the appointments link from dock 508, by selecting the Write Prescription link from user interface 500d or in any other suitable manner. In this example, the prescription could be for a drug (e.g., for treating an illness or for some other purpose) or for a vaccination.

For a drug prescription, user interface 500e includes a dropdown menu from which one or more drugs can be selected and, for each, a treatment window in which treatment instructions for administering the drug(s) (e.g., dosage, frequency, term, and the like) may be entered in free form. For vaccinations, user interface 500e includes a dropdown menu from which one or more vaccinations can be selected and added using the “Add a New Vaccination” button. User interface 500e includes a “Submit” button. In response to selection of the “Submit” button, animal management application 210 may communicate an update to animal management logic 312 to update digital animal records 112 for the associated horse with the added drugs/vaccinations.

As illustrated in FIG. 5F, user interface goof provides an interface for accepting or rejecting the requested appointment for Dancer. For example, a user may select either the Accept or Reject buttons to accept or reject, respectively, the requested appointment for Dancer. In certain embodiments, because the requested appointment for Dancer has not been accepted or rejected, the requested appointment for Dancer would appear in the appointments list 514 of user interface 500c when the Pending filter of filter banner 516 is applied. In response to selection of the Accept or Reject buttons, animal management application 210 may communicate an update to animal management logic 312 to update digital animal records 112 for the associated horse with appointment status. Additionally or alternatively, the Accept or Reject buttons could be for accepting or rejecting, respectively, a veterinarian video chat call, which if accepted (by touching the “Accept” button), may cause animal management application 210 to generated user interface 5ooj (described below).

Although not visible in the illustrated example of user interface 500f, scrolling down may reveal other links to other information about Dancer. For example, additional information (and potentially links to further details) for medical records, vaccinations, breeding details, health history, treatment history, and/or any other suitable information may be provided.

As illustrated in FIG. 5G, user interface 500g includes a vaccine list 518 of vaccinations that have been administered to a particular horse, along with a date on which the vaccine was administered.

As illustrated in FIG. 5H, user interface ooh includes radiography information, such as one or more X-ray images, for a particular horse. Additionally, user interface ooh includes a Radiography Notes section that may be used by the veterinarian or other user to input an analysis or other notes associated with a review of the X-ray images. In certain embodiments, the ability to view and share X-Ray images and/or videos may be implemented using DICOM technology.

As illustrated in FIG. 5I, user interface 500i includes surgery information for a previously executed or currently planned surgery. In the illustrated example, user interface 500i includes one or more X-ray images associated with the surgery. Additionally, user interface 500i includes a Surgery Notes section that may be used by the veterinarian or other user to input an analysis or other notes associated with the surgery.

As illustrated in FIG. 5J, user interface 500j includes a veterinarian video chat feature, which may correspond to the Vet Video Chat feature of user interface 400c described above with reference to FIG. 4C. User interface 500c may allow veterinarian to engage in a video chat with an owner or other individual caring for a horse without requiring the veterinarian to visit the farm or the owner (or other caretaker) to bring the animal to the veterinarian for evaluation. This may be particularly useful for initial consultations, issues that arise during non-business hours, or in other suitable scenarios. Furthermore, the veterinarian may be able to view certain portions of the horse to possibly provide an evaluation over the video chat.

As illustrated in FIG. 5K, user interface 500k includes a calendar and various appointment scheduling details for the veterinarian. As described above, in certain embodiments, user interface 500k may be reached using the Calendar link in dock 508. In the illustrated example, user interface 500k includes a calendar display 520, shown by month in this example, with an ability to change the displayed month using the arrows adjacent the October 2021 indication. A user may select a date (e.g., Oct. 9, 2021 is selected in the illustrated example), and an appointments list 522 may be displayed showing appointments for the selected date.

Appointments list 522 may indicate the total number of appointments (“3 Appointments” in the illustrated example), along with additional details of each appointment, one (for a horse named Knox) of which is shown due to the limited display area of the user device 102. Scrolling down may reveal the additional two appointments. Furthermore, the listed appointments from appointments list 522 may be selectable links that lead to a user interface for with details for the selected appointments.

User interface 500k includes an availability link 524 (shown as “Set Your Availability”), which may allow a user (e.g., a veterinarian) to set his or her availability for appointments, making only certain dates and times available if desired.

As illustrated in FIG. 5L, user interface 500l includes an availability list 526 that shows available appointments. In certain embodiments, animal management application 210 displays user interface 500l in response to user selection of availability link 524 from user interface 100k. In this example, user interface 500l includes a Set New Availability button ₅27, which may allow a user to modify availability list 526, such as by modifying existing listed availability or adding new availability.

As illustrated in FIG. 5M, user interface 500m includes dropdown menus for setting availability for appointments. In certain embodiments, animal management application 210 displays user interface 500m in response to user selection of the Set New Availability button 527 from user interface 500l. In the illustrated example, a user may set a range of times for a given date as being available. Selection of the Save button may cause animal management application 210 to communicate with animal management logic 312 to update the user's availability in digital animal records 112.

As illustrated in FIG. 5N, user interface soon may include settings for animal management application 210. As described above, in certain embodiments, user interface soon may be reached using the Settings link in dock 508. In the illustrated example, available settings include Profile Settings, Availability Schedule, My Wallet, Push Notification (to indicate whether animal management application 210 may push notifications to the user device 102 on which animal management application 210 is installed), About App (for displaying information about animal management application 210), Privacy Policy (to view the privacy policy for animal management application 210), and Sign Out (to sign out of the account shown in user interface 500a).

As illustrated in FIG. 5O, user interface s000 may include a My Wallet feature set for animal management application 210. As described above, in certain embodiments, user interface 500o may be reached by selecting the Total Earnings box of summary banner 504 in user interface 500a, using the My Wallet link in the settings display of user interface 500n, or in any other suitable manner.

In the illustrated example, the My Wallet features includes indicators of the total amount in the wallet (e.g., $480.00) and recent transactions. Additional transactions may be view using the “View All” link. User interface 500o also includes a Withdraw Amount button for initiating a withdrawal of a specified amount (e.g., to transfer to another bank account).

As illustrated in FIG. 5P, user interface 500p may include a display for completing a withdrawal of a certain amount from My Wallet. In certain embodiments, user interface 500p may be reached by selecting the Withdraw Amount button of user interface 500o. In the illustrated example, user interface 500p includes a list of available bank accounts, fields for adding a bank account, and a Withdraw button to initiate a withdrawal of any amount from My Wallet to a selected bank account.

As illustrated in FIG. 5Q, user interface 500q may include information about animal management application 210. In certain embodiments, user interface 500q may be reached by selecting the About App link from user interface soon (the Settings user interface). In the illustrated example, user interface 500q includes information regarding animal management application 210 (listed as Farmhand VET App), appointment details, and transactions information.

As illustrated in FIG. 5R, user interface 500r may include information regarding financial transactions associated with animal management application 210. In certain embodiments, user interface 500r may be reached by selecting the Transactions Information link from user interface 500q (the About App user interface). In the illustrated example, user interface 500q includes information regarding animal management application 210 (listed as Farmhand VET App), appointment details, and transactions information.

User interface 500r may include a transactions list 528, which lists zero or more transactions. The listed transactions may include payments received from clients of the veterinarian, transfers from My Wallet to a bank account or other recipient, or other suitable transactions, along with a date and amount of the transaction. In certain embodiments, selecting a particular transaction from transactions list 528 may cause animal management application 210 to display additional information about the particular transaction.

User interface 500r may include a filter banner 530, which may provide a number of options for filtering which transactions are shown in transactions list 528. In the illustrated example, the filtering options of filter banner 530 include Accepted (for displaying in transactions list 528 transactions that have been accepted), Pending (for displaying in transactions list 528 transactions that are pending (e.g., have been requested but have not been accepted or rejected)), and Rejected (for displaying in transactions list 528 transactions that have been rejected).

FIG. 6 illustrates example digital animal records 112, according to certain embodiments. In the illustrated example, digital animal records 112 are stored using storage unit 106. Digital animal records 112 include digital animal records 112a, digital animal records 112b, through digital animal records 112n, which may be referred to generally as digital animal records 112. Each set of digital animal records 112 may correspond to a different farm, a different corporate entity, or the like. In certain embodiments, digital animal records 112 for different entities (e.g., different corporate operations) may be stored in distinct storage units 106.

In this example, the animal data of digital animal records 112 (e.g., of digital animal records 112a) may be subdivided into animal inventory data 602, animal health data 604, animal certification data 606, and animal away care data 608. The particular subdivision of the data in digital animal records 112 that is illustrated and described is for example purposes only. Furthermore, although described separately, some of the data may overlap. Additionally, the digital animal records 112 may be organized in storage unit 106 in any suitable manner.

Animal inventory data 602 may include information for identifying and locating the animals, as well as certain factual information about the animals. For example, animal inventory data 602 may include identifiers for identifying the animals. As a particular example, an identifier of an animal may be a string of alphanumeric characters assigned to the animal. Additionally or alternatively, the identifier may be a code, such as a machine-readable image file or a physical representation thereof, examples of which include a quick response (QR) code and/or a barcode. In certain scenarios, the animal identifier may be originally assigned to the source animal when the source animal is entered into system 100 (e.g., into digital animal records 112). Although this disclosure contemplates any suitable component providing the animal identifier, in certain embodiments, processing system 104 (e.g., animal management logic 310) generates the animal identifier. The animal might or might not be tagged, branded, or otherwise physically labeled with the identifier. As another example, animal inventory data 602 may include biographical information for the animals. Such biographical information may include birthday, sex, and any other suitable biographical information. As another example, animal inventory data 602 may include location information identifying a location of the animal. The type of location information may depend on the type of animal as well as the manner in which a particular entity organizes animals. In a particular example of horses, the location information may include a suitable combination of Farm, Stable, and Stall.

Animal health data 604 may include information regarding the health of the animals being managed using system 100. For example, animal health data 604 may include medical records for the animals. As another example, animal health data 604 may include breeding information for the animals. As another example, animal health data 604 may include vaccination records for the animals. As another example, animal health data 604 may include medications for the animals. As another example, animal health data 604 may include historical, current, or future health treatments for the animals. As another example, animal health data 604 may include health reminders for the animals. Such health reminders may include reminders to administer certain medications, reminders for veterinarian visits, and the like.

Animal certification data 606 may include information regarding the registrations and certifications that have been completed for each animal. An example certificate for a particular horse may include a membership registration card for a particular agency (e.g., the AQHA), a certificate of registration with a particular agency (e.g., the AQHA), an inspection form (e.g., an Equine Inspection Anemia Test Form, such as a so-called Coggins report), or other suitable documents.

Animal away care data 608 may include information regarding the tasks, task details, and respective responsible entities for one or more animals to be performed while the animal owner/caretaker is away from the farm. For example, the tasks could include feeding each horse, the task details could include a feeding schedule and food amount for each horse, and one or more responsible entities may be identified for performing the feeding(s). As another example, the tasks could include, for each of one or more horses, administering one or more medications. The task details may include dosage amounts and schedules, and a responsible entity may be identified for administering the drug.

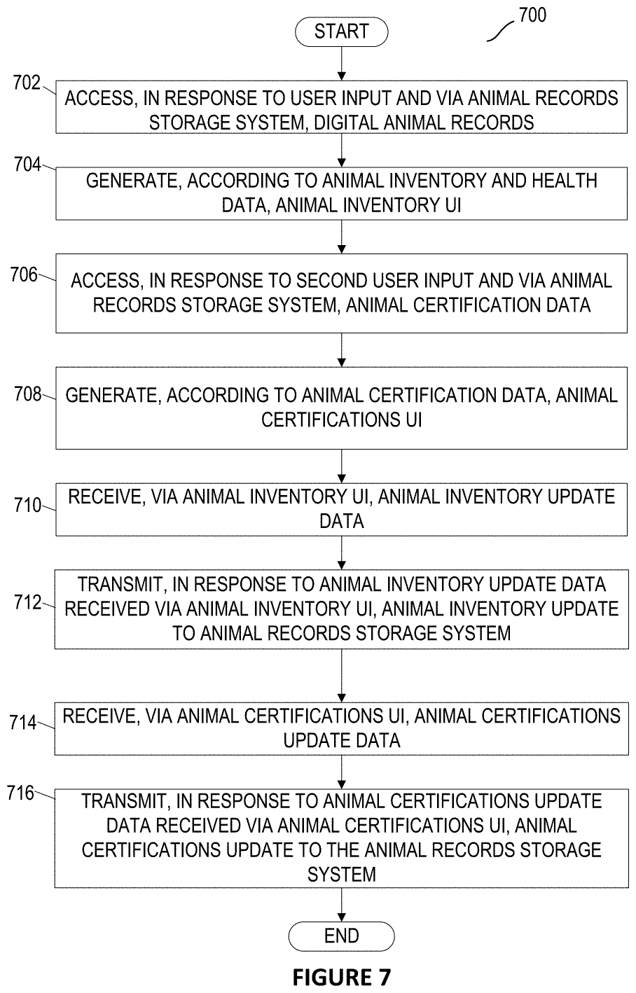

FIG. 7 illustrates an example method 700 for managing digital animal records for managing animal health, according to certain embodiments. In certain embodiments, the operations of method 700 may be performed by animal management application 210 running on user device 102. In certain embodiments, the animals being managed using system 100 may be horses, however, this disclosure contemplates managing any suitable type or types of animals using system 100 and method 700. Additionally, in certain embodiments, the features described with reference to method 700 may be particularly applicable to a user no who is an owner/caretaker of the animals (e.g., a horse owner), though this disclosure contemplates any user for method 700.

At step 702, animal management application 210 may access, in response to user input using user device 102 and via an animal records storage system (e.g., storage unit 106), digital animal records 112 for a plurality of animals. Digital animal records 112 may store animal data for the animals, and the animal data may include animal inventory data, animal certification data, and animal health data.

At step 704, animal management application 210 may generate, according to the animal inventory data and the animal health data, an animal inventory user interface. The animal inventory user interface may include identifying information of a first animal being managed using system 100 and health information of the first animal.

In certain embodiments, a user interface generated by animal management application 210 includes a link for adding a new animal to the inventory of managed animals. In such an example, animal management application 210 may be configured to receive animal inventory data for the new animal and transmit the animal inventory data for the new animal for storage in the animal records storage system (e.g., in digital animal records 112 of storage unit 106).

At step 706, animal management application 210 may access, in response to user input from user device 102 and via the animal records storage system (e.g., storage unit 106), animal certifications data.

At step 708, animal management application 210 may generate, according to the animal certification data, an animal certifications user interface for managing the animal certifications of one or more of the animals. In certain embodiments, the animal certifications user interface may include certifications of the first animal.

At step 710, animal management application 210 may receive, via the animal inventory user interface displayed on user device 102, animal inventory update data, and at step 712, animal management application 210 may transmit, in response to the animal inventory update data received via the animal inventory user interface, an animal inventory update to the animal records storage system (e.g., storage unit 106) for updating digital animal records 112 (e.g., animal inventory data).

At step 714, animal management application 210 may receive, via the animal certifications user interface displayed on user device 102, animal certifications update data; and at step 716, animal management application 210 may transmit, in response to the animal certifications update data received via the animal certifications user interface, an animal certifications update to the animal records storage system (e.g., storage unit 106) for updating digital animal records 112 (e.g., animal certifications data).

In certain embodiments, animal management application 210 may generate a dashboard user interface screen that includes selectable links. Each selectable link may correspond to an associated feature and data set for managing animal health of the managed animal inventory. For example, a first selectable link may include a link to the dashboard user interface. As another example, a second selectable link may include a link to an away care user interface. As another example, a third selectable link may include a link to a veterinarian video chat user interface. As another example, a fourth selectable link may include a link to a health reminders user interface. As another example a fifth selectable link may include a link to a breeding records user interface. As another example, a sixth selectable link may include a link to a medical records user interface. As another example, a seventh selectable link may include a link to a racing user interface.

In certain embodiments, animal management application 210 is configured to access, via the animal records storage system (e.g., storage unit 106), breeding information for a subset of the animals, and to generating, according to the breeding information, a breeding user interface. As examples, the breeding user interface may include breeding information for the subset of animals or the breeding user interface may include breeding details for a particular animal of the subset of animals.

In certain embodiments, animal management application 210 is configured to access, in response to user input, animal away care data for one or more animals. The animal away care data may include tasks, task details, and a respective responsible entity for performing each task according to the task details for that task. Animal management application 210 may be configured to display, in response to accessing the animal away care data, at least a portion of the animal away care data for the one or more animals, and to generate, for each of the one or more animals, a respective notification that the tasks for the animal have been completed.

In certain embodiments, animal management application 210 is configured to receive user input requesting a video chat with a selected veterinarian; initiate, in response to the user input, the video chat with the selected veterinarian; and display a video chat user interface for conducting the video chat with the selected veterinarian.

In certain embodiments, animal management application 210 is configured to access, in response to user input, the animal health data for the first animal. The animal health data may include medical records for the first animal. Animal management application 210 may be configured to display, in response to the user input, at least a portion of the medical records for the first animal in a medical records user interface.

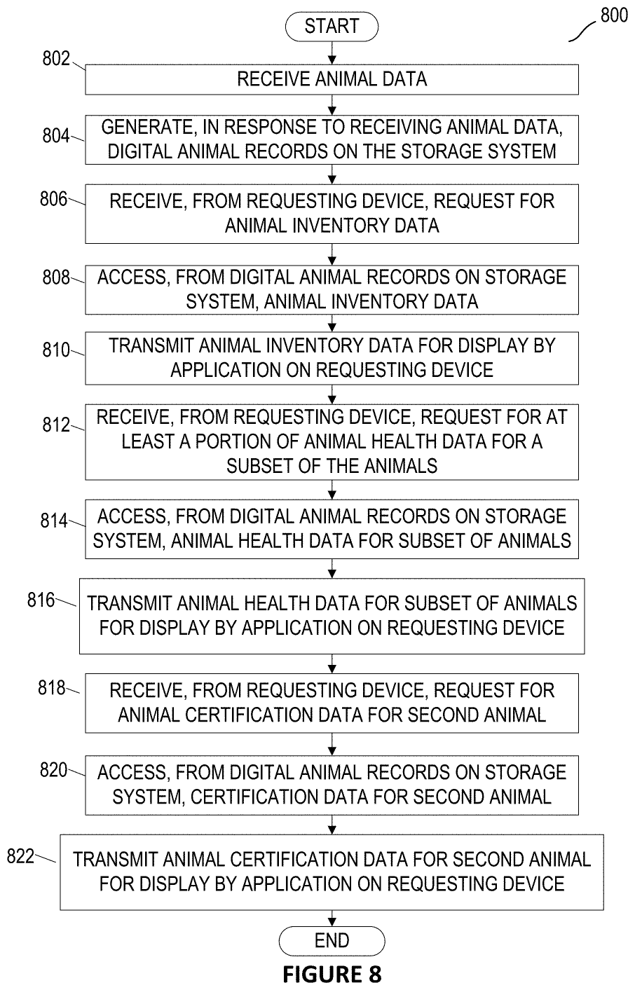

FIG. 8 illustrates an example method 800 for managing digital animal records for managing animal health, according to certain embodiments. In certain embodiments, the operations of method 800 may be performed by animal management logic 310 running on processing system 104. In certain embodiments, the animals being managed using system 100 may be horses, however, this disclosure contemplates managing any suitable type or types of animals using system 100 and method 800.

At step 802, animal management logic 310 may receive animal data for animals. The animal data may include animal inventory data, animal certification data, and animal health data. At step 804, animal management logic 310 may generate, in response to receiving the animal data for the animals, digital animal records 112 on the storage system (e.g., storage unit 106). Digital animal records 112 may include the animal data for the animals.

At step 806, animal management logic 310 may receive, from a requesting user device 102, a request for the animal inventory data for a first animal. At step 808, animal management logic 310 may access, from digital animal records 112 on the storage system (e.g., storage unit 106), the animal inventory data for the first animal, and at step 810, animal management logic 310 may transmit, to the requesting user device 102, the animal inventory data for display by an application (e.g., animal management application 210) running on the requesting user device 102. In certain embodiments, the animal inventory data includes the following information for the managed inventory of animals: an animal identifier; animal type; and location information.

At step 812, animal management logic 310 may receive, from a requesting user device 102, a request for at least a portion of the animal health data for a subset of the animals. Of course the user device 102 from which the request received at step 812 is received may be the same or different as the user device 102 from which the request received at step 806 is received. At step 814, animal management logic 310 may access, from digital animal records 112 on the storage system (e.g., storage unit 106), the animal health data for the subset of the animals, and at step 816, animal management logic 310 may transmit, to the requesting user device 102, the animal health data for the subset of the animals for display by an application (e.g., animal management application 210) running on the requesting user device 102. In certain embodiments, the animal health data includes the following information for the managed inventory of animals: digital medical records; vaccination information; breeding information; and treatment information. In certain embodiments, the animal health data includes a schedule of health events and animal management logic 310 is configured to communicate a reminder of a health event to an application (e.g., animal management application 210) running on a requesting user device 102.

At step 818, animal management logic 310 may receive, from a requesting user device 102, a request for at least a portion of the animal certification data for a second animal (which may be the same or different than the first animal of step 806). Of course the user device 102 from which the request received at step 818 is received may be the same or different as the user devices 102 from which the requests received at steps 806 and 812 are received. At step 820, animal management logic 310 may access, from digital animal records 112 on the storage system (e.g., storage unit 106), the at least a portion of the animal certification data for the second animal, and at step 822, animal management logic 310 may transmit, to the requesting user device 102, the at least a portion of the animal certification data for the second animal for display by an application (e.g., animal management application 210) running on the requesting user device 102.

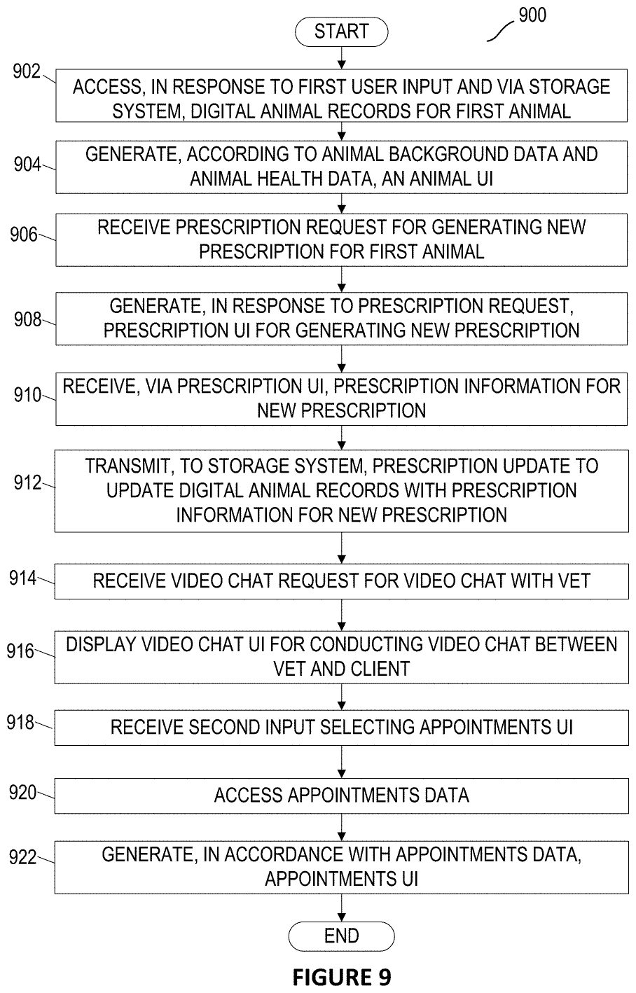

FIG. 9 illustrates an example method 900 for managing digital animal records for managing animal health, according to certain embodiments. In certain embodiments, the operations of method 900 may be performed by animal management application 210 running on user device 102. In certain embodiments, the animals being managed using system 100 may be horses, however, this disclosure contemplates managing any suitable type or types of animals using system 100 and method 900. Additionally, in certain embodiments, the features described with reference to method 900 may be particularly applicable to a user no who is a veterinarian, though this disclosure contemplates any user for method 900. To perform method 900, animal management application may interact with one or more of animal management logic 310, digital animal data 112, and/or digital veterinarian data 312.

At step 902, animal management application 210 may access, in response to user input using user device 102 and via an animal records storage system (e.g., storage unit 106) that stores digital animal records 112 for a plurality of animals, digital animal records 112 for a first animal. Digital animal records 112 may store animal data for the animals, and the animal data may include animal background data and animal health data.

At step 904, animal management application 210 may generate, according to the animal background data and the animal health data, an animal user interface. The animal user interface may include animal background information for the first animal being managed using system 100 and animal health information for the first animal.

In certain embodiments, a user interface generated by animal management application 210 includes a link for adding a new animal to the inventory of managed animals. In such an example, animal management application 210 may be configured to receive animal inventory data for the new animal and transmit the animal inventory data for the new animal for storage in the animal records storage system (e.g., in digital animal records 112 of storage unit 106).

At step 906, animal management application 210 may receive, via the animal inventory user interface displayed on user device 102, a prescription request for generating a new prescription for the first animal, and at step 908, animal management application 210 may generate, in response to the prescription request, a prescription user interface for generating the new prescription for the first animal. In certain embodiments, the new prescription may be for a medication or a vaccination.

At step 910, animal management application 210 may receive, via the prescription user interface (e.g., user interface 500e), prescription information for the new prescription. At step 912, animal management application 210 may transmit, to the storage system (e.g., storage unit 106), a prescription update to update digital animal records 112 with the prescription information for the new prescription.

At step 914, animal management application 210 may receive a video chat request for a video chat with a veterinarian, and at step 916 animal management application 210 may display a video chat user interface for conducting the video chat between the veterinarian and a client (e.g., a horse owner).

At step 918, animal management application 210 may receiving a second input selecting a selectable link to an appointments user interface, and at step 920, animal management application 210 may access, in response to receiving the second input and via the storage system (e.g., storage unit 106), appointments data. At step 922, animal management application 210 may generate, in accordance with the appointments data, an appointments user interface. In certain embodiments, the appointments user interface may include filters for filtering a listing of appointments and the listing of appointments according to a selection of one or more of the filters.

In certain embodiments, animal management application 210 may generate a dashboard user interface screen that includes selectable links. In certain embodiments, these selectable links may be implemented, in part, using a dock (e.g., dock 508). Each selectable link may correspond to an associated feature and data set for managing animal health of the managed animal inventory. For example, a first selectable link may include a link to the dashboard user interface. As another example, a second selectable link may include a link to an appointments user interface. As another example, a third selectable link may include a link to a medical records user interface. As another example, a fourth selectable link may include a link to a calendar user interface. As another example a fifth selectable link may include a link to a settings user interface.

In certain embodiments, the first input of step 902 is a selection of the selectable link to the medical records user interface such that the animal user interface (generated at step 904) is the medical records user interface, as generated for the first animal. The medical records user interface may include a first link, selection of which initiates generation of a vaccination user interface that is configured to list vaccinations for the first animal; a second link, selection of which initiates generation of a radiograph user interface that is configured to display an image of an X-ray for the first animal; and a third link, selection of which initiates generation of a surgery user interface that is configured to display notes or images associated with a surgery for the first animal.

Although the present disclosure describes or illustrates particular operations as occurring in a particular order, the present disclosure contemplates any suitable operations occurring in any suitable order. Moreover, the present disclosure contemplates any suitable operations being repeated one or more times in any suitable order. Although the present disclosure describes or illustrates particular operations as occurring in sequence, the present disclosure contemplates any suitable operations occurring at substantially the same time, where appropriate. Any suitable operation or sequence of operations described or illustrated herein may be interrupted, suspended, or otherwise controlled by another process, such as an operating system or kernel, where appropriate. The acts can operate in an operating system environment or as stand-alone routines occupying all or a substantial part of the system processing.

Certain embodiments may provide none, some, or all of the following technical advantages.

Certain embodiments provide a highly intuitive user interface for viewing, sharing, updating, or otherwise interacting with animal data stored in digital animal records, including animal inventory data and animal health data. For example, through a software application running on a mobile device, farmers and other workers associated with a livestock operation may enter animal data for one or more groups of animals being raised by the livestock operation to establish digital data records for those animals. Those digital data records include animal inventory data and animal health data. The software application may provide a variety of interfaces for interacting with the digital data records to facilitate managing the health of the animals being raised by the livestock operation.

Certain embodiments may save time by reducing or eliminating hand-written health records through a partial or complete digital solution. Certain embodiments may reduce costs by allowing decision makers to use real-time data to make timely, potentially improved management decisions. Certain embodiments allow farmers and associated users to communicate with a veterinarian from virtually anywhere at any time. Certain embodiments may improve animal health. Certain embodiments may cut veterinary costs through better routine health programs. Certain embodiments provide a veterinary electronic medical record for an animal or group of animals.

Through the user interfaces implemented according to certain embodiments of this disclosure, certain embodiments are able to display a limited set of information in an organized manner, resulting in an improved user interface, to facilitate managing the health of the livestock of an organization. User interfaces provided by certain embodiments of this disclosure may allow a user to more quickly access data and applications in electronic devices, improving the speed of a user's navigation through various views and windows.

Certain embodiments provide a digital animal record that includes animal health data (e.g., a digital medical record) and/or registration/certification documentation that can travel with the animal and be presented as appropriate at animal shows, auctions, races, or other suitable events. Indeed, with respect to horses, certain regulatory agencies may require presentation of such documentation for a horse to be eligible to participate in the event. Rather than bringing physical papers, certain embodiments of this disclosure provide an ability to quickly and easily present electronic versions of those medical records in an organized manner using the intuitive user interfaces provided by the animal management application 210, animal management logic 310, and associated digital animal records 112.

Certain embodiments provide a veterinarian application suitable for use by a veterinarian, potentially in conjunction with veterinary clients who use the above-described animal applications. The veterinarian application may be a mobile software platform for veterinarians. The platform may include vaccination/dewormer reminders/records, general health/medical digital record, vet video chat, breeding records, registration certificates, horse (or another animal type, if appropriate) profiles, and financial transaction functionality. In certain embodiments, the veterinarian application interacts with at least a portion of the same digital animal records (e.g., digital animal records 112) that a horse owner version of the application interacts with, which may help ensure that the veterinarian and horse owner are working with the same information and are able to see, in real time, updates to those digital animal records.

Example embodiments of this disclosure are summarized here. Other embodiments can also be understood from the entirety of the specification as well as the claims filed herein.

Example 1: A non-transitory computer-readable storage medium stores a program for execution by one or more processors. The program includes instructions for accessing, in response to a first user input and via an equine records storage system, digital equine records for equines that include equine data for the equines. The equine data includes equine inventory data, certification data, and health data. The program includes instructions for generating, according to the equine inventory data and health data, an equine inventory user interface that includes identifying information of a first equine and health information for the first equine. The program includes instructions for accessing, in response to a second user input and via the storage system, the equine certification data, and generating, according to the equine certification data, an equine certifications user interface that includes certifications of the first equine. The program includes instructions for receiving, via the equine inventory user interface, equine inventory update data, and, in response, transmitting an equine inventory update to the storage system for updating the equine inventory data.

Example 2: The non-transitory computer-readable medium of Example 1, where the program further includes instructions for: receiving, via the equine certifications user interface, equine certifications update data; and transmitting, in response to the equine certifications update data received via the equine certifications user interface, an equine certifications update to the equine records storage system for updating the equine certifications data.

Example 3: The non-transitory computer-readable medium of any one of Examples 1-2, where the program further includes instructions for generating a dashboard user interface screen that includes selectable links, each selectable link of the selectable links corresponding to an associated feature and data set for managing equine health of the equines.

Example 4: The non-transitory computer-readable storage medium of Example 3, where: a first selectable link of the selectable links includes a link to an away care user interface; a second selectable link of the selectable links includes a link to a veterinarian video chat user interface; a third selectable link of the selectable links includes a link to a health reminders user interface; a fourth selectable link of the selectable links includes a link to a breeding records user interface; a fifth selectable link of the selectable links includes a link to a medical records user interface; and a sixth selectable link of the selectable links includes a link to a racing user interface.

Example 5: The non-transitory computer-readable storage medium of any one of Examples 1-4, where the program further includes instructions for: accessing, via the equine records storage system, breeding information for a subset of the equines; and generating, according to the breeding information, a breeding user interface.

Example 6: The non-transitory computer-readable storage medium of Example 7, where: the breeding user interface includes breeding information for the subset of the equines; or the breeding user interface includes breeding details for a particular equine of the subset of the equines.

Example 7: The non-transitory computer-readable medium of any one of Examples 1-6, where the program further includes instructions for: accessing, in response to third user input, equine away care data for one or more horses, the equine away care data including tasks, task details, and a respective responsible entity for performing each task according to the task details for that task; displaying, in response to accessing the equine away care data, at least a portion of the equine away care data for the one or more horses; and generating, for each of the one or more horses, a respective notification that the tasks for the horse have been completed.

Example 8: The non-transitory computer-readable medium of any one of Examples 1-7, where the program further includes instructions for: receiving third user input requesting a video chat with a selected veterinarian; initiating, in response to third user input, the video chat with the selected veterinarian; and displaying a video chat user interface for conducting the video chat with the selected veterinarian.

Example 9: The non-transitory computer-readable medium of any one of Examples 1-8, where the program further includes instructions for: accessing, in response to third user input, the equine health data for the first equine, the equine health data including medical records for the first equine; and displaying, in response to the third user input, at least a portion of the medical records for the first equine in a medical records user interface.

Example 10: In certain embodiments, a system includes a storage system, one or more processors, and a non-transitory computer-readable storage medium storing a program to be executed by the one or more processors. The program includes instructions for receiving equine data for equines that includes equine inventory data, equine certification data, and equine health data. The program includes instructions for generating, in response to receiving the equine data for the equines, digital equine records on the storage system, the digital equine records including the equine data for the equines. The program includes instructions for receiving, from a first requesting device, a request for at least a portion of the equine inventory data for a first equine; accessing, from digital equine records on the storage system, the equine inventory data for the first equine; and transmitting, to the first requesting device, the at least a portion of the equine inventory data for the first equine for display by an application on the first requesting device. The program includes instructions for receiving, from a second requesting device, a request for at least a portion of the equine certification data for a second equine; accessing, from the digital equine records on the storage system, the equine certification data for the second equine; and transmitting, to the second requesting device, the at least a portion of the equine certification data for the second equine for display by an application on the second requesting device.

Example 11: The system of Example 10, where: the first requesting device and the second requesting device are a same device; and the first equine and the second equine are a same equine.

Example 12: The system of any one of Examples 10-11, where the program further includes instructions for: receiving, from a third requesting device, a request for at least a portion of the equine health data for a third equine of the equines; accessing, from the digital equine records on the storage system, the equine health data for the third equine; and transmitting, to the third requesting device, the at least a portion the equine health data for the third equine for display by an application on the second requesting device, where the third requesting device is a same requesting device than one or more of the first requesting device or the second requesting device, and where the third equine is a same equine as one or more of the first equine or the second equine.

Example 13: The system of Example 12, where: the equine health data includes a schedule of health events; and the program further includes instructions for communicating a reminder of a health event to an application on the third requesting device.

Example 14: The system of any one of Examples 10-13, where: the equine inventory data includes the following information for the equines: an equine identifier; equine type; and location information; and the equine health data includes the following information for the equines: digital medical records; vaccination information; breeding information; and treatment information.

Example 15: In certain embodiments, a non-transitory computer-readable storage medium stores a program for execution by one or more processors. The program includes instructions for accessing, in response to a first user input and via a storage system storing digital animal records for animals, the digital animal records for a first animal, the digital animal records including animal background data and animal health data. The program includes instructions for generating, according to the animal background data and the animal health data, an animal user interface that includes background information and animal health information for the first animal. The program includes instructions for receiving a prescription request for generating a new prescription for the first animal; and generating, in response to the prescription, a prescription user interface for generating the new prescription for the first animal, the new prescription being for a medication or a vaccination. The program includes instructions for receiving, via the prescription user interface, prescription information for the new prescription; and transmitting, to the storage system, a prescription update to update the digital animal records with the prescription information for the new prescription.

Example 16: The non-transitory computer-readable medium of Example 15, where the program further includes instructions for generating a dashboard user interface screen that includes selectable links, each selectable link corresponding to an associated feature and data set for managing animal health of the animals.

Example 17: The non-transitory computer-readable medium of Example 16, where: first selectable link of the selectable links includes a link to the dashboard user interface; a second selectable link of the selectable links includes a link to an appointments user interface; a third selectable link of the selectable links includes a link to a medical records user interface; a fourth selectable link of the selectable links includes a link to a calendar user interface; and a fifth selectable link of the selectable links includes a link to a settings user interface.

Example 18: The non-transitory computer-readable medium of any one of Examples 16-17, where: the selectable links include a selectable link to an appointments user interface; and the program further includes instructions for: receiving a second input selecting the selectable link to the appointments user interface; accessing, in response to receiving the second input and via the storage system, appointments data; and generating, in accordance with the appointments data, the appointments user interface, the appointments user interface including: filters for filtering a listing of appointments; and the listing of appointments according to a selection of a filter of the filters.

Example 19: The non-transitory computer-readable medium of any one of Examples 16-18, where: the selectable links include a selectable link to a medical records user interface; the first input includes a selection of the selectable link to the medical records user interface such that the animal user interface is the medical records user interface, as generated for the first animal; the medical records user interface includes: a first link, selection of which initiates generation of a vaccination user interface that is configured to list vaccinations for the first animal; a second link, selection of which initiates generation of a radiograph user interface that is configured to display an image of an X-ray for the first animal; and a third link, selection of which initiates generation of a surgery user interface that is configured to display notes or images associated with a surgery for the first animal.

Example 20: The non-transitory computer-readable medium of any one of Examples 15-20, where the program further includes instructions for: receiving a video chat request for a video chat with a veterinarian; and displaying a video chat user interface for conducting the video chat between the veterinarian and a client.

In certain embodiments, a system includes one or more processors and a non-transitory computer-readable storage medium storing a program to be executed by the processor. The program includes instructions for: generating a user interface comprising fields for managing animal health associated with one or more animals; generate a digital data record for the one or more animals, the digital data record comprising health records associated with the one or more animals; and causing the digital data record to be stored.

In certain embodiments, a non-transitory computer-readable storage medium stores a program to be executed by one or more processors. The program includes instructions for: generating a user interface comprising fields for managing animal health associated with one or more animals; generating a digital data record for the one or more animals, the digital data record comprising health records associated with the one or more animals; and causing the digital data record to be stored.

In certain embodiments, a method includes: generating a user interface comprising fields for managing animal health associated with one or more animals; generating a digital data record for the one or more animals, the digital data record comprising health records associated with the one or more animals; and causing the digital data record to be stored.

An example processing system, according to certain embodiments of this disclosure, may be configured to perform methods/operations described in this disclosure, and may be installed in a host device. The processing system may include a processor, a memory, and interfaces. The processor may be any component or collection of components adapted to perform computations and/or other processing related tasks, and the memory may be any component or collection of components adapted to store programming and/or instructions for execution by processor. In an embodiment, the memory includes a non-transitory computer readable medium. The computer-readable non-transitory media includes all types of computer readable media, including magnetic storage media, optical storage media, and solid-state storage media and specifically excludes signals. It should be understood that the software can be installed in and sold with the device. Alternatively, the software can be obtained and loaded into the device, including obtaining the software via a disc medium or from any manner of network or distribution system, including, for example, from a server owned by the software creator or from a server not owned but used by the software creator. The software can be stored on a server for distribution over the Internet, for example.

While this disclosure has been described with reference to illustrative embodiments, this description is not intended to be construed in a limiting sense. Various modifications and combinations of the illustrative embodiments, as well as other embodiments of the disclosure, will be apparent to persons skilled in the art upon reference to the description. It is therefore intended that the appended claims encompass any such modifications or embodiments.

What is claimed is:

1. A non-transitory computer-readable storage medium storing a program for execution by one or more processors, the program including instructions for:
accessing, in response to a first user input and via an equine records storage system, digital equine records for a plurality of equines, the digital equine records comprising equine data for the plurality of equines, the equine data comprising equine inventory data, equine certification data, and equine health data;
generating, according to the equine inventory data and the equine health data, an equine inventory user interface, wherein the equine inventory user interface comprises identifying information of a first equine of the plurality of equines and health information for the first equine;
accessing, in response to a second user input and via the equine records storage system, the equine certification data;
generating, according to the equine certification data, an equine certifications user interface, wherein the equine certifications user interface comprises one or more certifications of the first equine;
receiving, via the equine inventory user interface, equine inventory update data; and
transmitting, in response to the equine inventory update data received via the equine inventory user interface, an equine inventory update to the equine records storage system for updating the equine inventory data.

2. The non-transitory computer-readable medium of Claim i, wherein the program further includes instructions for:
receiving, via the equine certifications user interface, equine certifications update data; and
transmitting, in response to the equine certifications update data received via the equine certifications user interface, an equine certifications update to the equine records storage system for updating the equine certifications data.

3. The non-transitory computer-readable medium of claim 1, wherein the program further includes instructions for generating a dashboard user interface screen that comprises a plurality of selectable links, each selectable link of the plurality of selectable links corresponding to an associated feature and data set for managing equine health of the plurality of equines.

4. The non-transitory computer-readable storage medium of claim 3, wherein:
a first selectable link of the plurality of selectable links comprises a link to an away care user interface;
a second selectable link of the plurality of selectable links comprises a link to a veterinarian video chat user interface;
a third selectable link of the plurality of selectable links comprises a link to a health reminders user interface;
a fourth selectable link of the plurality of selectable links comprises a link to a breeding records user interface;
a fifth selectable link of the plurality of selectable links comprises a link to a medical records user interface; and
a sixth selectable link of the plurality of selectable links comprises a link to a racing user interface.

5. The non-transitory computer-readable storage medium of claim 1, wherein the program further includes instructions for:
accessing, via the equine records storage system, breeding information for a subset of the plurality of equines; and
generating, according to the breeding information, a breeding user interface.

6. The non-transitory computer-readable storage medium of claim 7, wherein:
the breeding user interface comprises breeding information for the subset of the plurality of equines; or
the breeding user interface comprises breeding details for a particular equine of the subset of the plurality of equines.

7. The non-transitory computer-readable medium of claim 1, wherein the program further includes instructions for:
accessing, in response to third user input, equine away care data for one or more horses, the equine away care data comprising tasks, task details, and a respective responsible entity for performing each task according to the task details for that task;
displaying, in response to accessing the equine away care data, at least a portion of the equine away care data for the one or more horses; and
generating, for each of the one or more horses, a respective notification that the tasks for the horse have been completed.

8. The non-transitory computer-readable medium of claim 1, wherein the program further includes instructions for:
receiving third user input requesting a video chat with a selected veterinarian;
initiating, in response to third user input, the video chat with the selected veterinarian; and
displaying a video chat user interface for conducting the video chat with the selected veterinarian.

9. The non-transitory computer-readable medium of claim 1, wherein the program further includes instructions for:
accessing, in response to third user input, the equine health data for the first equine, the equine health data comprising medical records for the first equine; and
displaying, in response to the third user input, at least a portion of the medical records for the first equine in a medical records user interface.

10. A system, comprising:
a storage system;
one or more processors;
a non-transitory computer-readable storage medium storing a program to be executed by the one or more processors, the program including instructions for:
receiving equine data for a plurality of equines, the equine data comprising equine inventory data, equine certification data, and equine health data;
generating, in response to receiving the equine data for the plurality of equines, digital equine records on the storage system, the digital equine records comprising the equine data for the plurality of equines;
receiving, from a first requesting device, a request for at least a portion of the equine inventory data for a first equine of the plurality of equines;
accessing, from digital equine records on the storage system, the equine inventory data for the first equine;
transmitting, to the first requesting device, the at least a portion of the equine inventory data for the first equine for display by an application on the first requesting device;
receiving, from a second requesting device, a request for at least a portion of the equine certification data for a second equine of the plurality of equines;
accessing, from the digital equine records on the storage system, the equine certification data for the second equine; and
transmitting, to the second requesting device, the at least a portion of the equine certification data for the second equine for display by an application on the second requesting device.

11. The system of claim 10, wherein:
the first requesting device and the second requesting device are a same device; and
the first equine and the second equine are a same equine.

12. The system of claim 10, wherein the program further includes instructions for:
receiving, from a third requesting device, a request for at least a portion of the equine health data for a third equine of the plurality of equines;
accessing, from the digital equine records on the storage system, the equine health data for the third equine; and
transmitting, to the third requesting device, the at least a portion the equine health data for the third equine for display by an application on the second requesting device, wherein the third requesting device is a same requesting device than one or more of the first requesting device or the second requesting device, and wherein the third equine is a same equine as one or more of the first equine or the second equine.

13. The system of claim 12, wherein:
the equine health data comprises a schedule of health events; and
the program further includes instructions for communicating a reminder of a health event to an application on the third requesting device.

14. The system of claim 10, wherein:
the equine inventory data comprises the following information for the plurality of equines:
an equine identifier;
equine type; and
location information; and
the equine health data comprises the following information for the plurality of equines:
digital medical records;
vaccination information;
breeding information; and
treatment information.

15. A non-transitory computer-readable storage medium storing a program for execution by one or more processors, the program including instructions for:
accessing, in response to a first user input and via a storage system storing digital animal records for a plurality of animals, the digital animal records for a first animal of the plurality of animals, the digital animal records comprising animal background data and animal health data;
generating, according to the animal background data and the animal health data, an animal user interface, wherein the animal user interface comprises background information and animal health information for the first animal;
receiving a prescription request for generating a new prescription for the first animal;
generating, in response to the prescription request, a prescription user interface for generating the new prescription for the first animal, the new prescription being for a medication or a vaccination;
receiving, via the prescription user interface, prescription information for the new prescription; and
transmitting, to the storage system, a prescription update to update the digital animal records with the prescription information for the new prescription.

16. The non-transitory computer-readable medium of claim 15, wherein the program further includes instructions for generating a dashboard user interface screen that comprises a plurality of selectable links, each selectable link of the plurality of selectable links corresponding to an associated feature and data set for managing animal health of the plurality of animals.

17. The non-transitory computer-readable medium of claim 16, wherein:
a first selectable link of the plurality of selectable links comprises a link to the dashboard user interface;
a second selectable link of the plurality of selectable links comprises a link to an appointments user interface;
a third selectable link of the plurality of selectable links comprises a link to a medical records user interface;
a fourth selectable link of the plurality of selectable links comprises a link to a calendar user interface; and
a fifth selectable link of the plurality of selectable links comprises a link to a settings user interface.

16. on-transitory computer-readable medium of claim 16, wherein:
the plurality of selectable links comprises a selectable link to an appointments user interface; and
the program further includes instructions for:
receiving a second input selecting the selectable link to the appointments user interface;
accessing, in response to receiving the second input and via the storage system, appointments data; and
generating, in accordance with the appointments data, the appointments user interface, the appointments user interface comprising:
a plurality of filters for filtering a listing of appointments; and
the listing of appointments according to a selection of a filter of the plurality of filters.

19. The non-transitory computer-readable medium of claim 16, wherein:
the plurality of selectable links comprises a selectable link to a medical records user interface;
the first input comprises a selection of the selectable link to the medical records user interface such that the animal user interface is the medical records user interface, as generated for the first animal;
the medical records user interface comprises:
a first link, selection of which initiates generation of a vaccination user interface that is configured to list vaccinations for the first animal;
a second link, selection of which initiates generation of a radiograph user interface that is configured to display an image of an X-ray for the first animal; and
a third link, selection of which initiates generation of a surgery user interface that is configured to display notes or images associated with a surgery for the first animal.

20 . The non-transitory computer-readable medium of claim 15, wherein the program further includes instructions for:
receiving a video chat request for a video chat with a veterinarian; and
displaying a video chat user interface for conducting the video chat between the veterinarian and a client.
